(19) United States
(12) Patent Application Publication
(10) Pub. No.: US 2024/0091254 A1
(43) Pub. Date: Mar. 21, 2024

(54) WNT AGONISTS FOR PREVENTION OF CANCER

(71) Applicant: ACADEMISH MEDISCH CENTRUM, Amsterdam (NL)

(72) Inventors: Sanne Marcella Maria Van Neerven, Amsterdam (NL); Louis Vermeulen, Amsterdam (NL)

(21) Appl. No.: 18/554,382

(22) PCT Filed: Apr. 12, 2022
(86) PCT No.: PCT/EP2022/059802

§ 371 Date: Oct. 6, 2023

(30) Foreign Application Priority Data
Apr. 12, 2021 (GB) 2105212.1

(51) Int. Cl. A61K 33/14; A61K 45/06; A61P 35/00

(52) U.S. Cl. CPC A61K 33/14; A61K 45/06; A61P 35/00

(57) ABSTRACT

There is provided a Wnt agonist for use in preventing gastrointestinal tract cancer in a subject, wherein the subject has a genetic predisposition to gastrointestinal tract cancer such as FAP as well as a Wnt agonist for preventing or inhibiting the formation of intestinal pre-cancerous lesions or intestinal adenomas.

### FIELD OF THE INVENTION

The present invention relates to the use of a Wnt agonist in therapy. More specifically, the present invention relates to the use of a Wnt agonist for use in preventing cancer in subjects predisposed to gastrointestinal polyps and for use in preventing or inhibiting the formation of intestinal pre-cancerous lesions or intestinal adenomas.

### BACKGROUND OF THE INVENTION

Colorectal cancer (CRC) formation is a prime example of stepwise cancer development. It is thought that the majority of CRCs are initiated by permanent activation of the Wnt pathway, often through mutations in tumour suppressor gene APC that occur within the stem cell pool⁴. Subsequently, the continuously on-going neutral replacement events between a relatively small number of Intestinal Stem Cells (ISCs) residing in the crypt bottom are distorted in the affected crypt, and Apc^{-/-} ISCs display a positive bias to replace their Apc-proficient neighbours³. As a result, Apc-mutant ISCs and their offspring have an increased probability to fully populate the crypt in which they arise, and initiate tumour formation. Apc mutations induce increased proliferation, prevent cell death, and block differentiation in the intestine^{5,6}. All these features might contribute to the increased relative fitness of Apc-mutant cells, but given the inherent difficulty of directly targeting the Wnt signalling cascade, to date, these insights have not resulted in more effective therapies or in novel preventive strategies for CRC.

Dow, Lukas E., et al. “APC restoration promotes cellular differentiation and re-establishes crypt homeostasis in colorectal cancer.” Cell 161.7 (2015): 1539-1552 discloses that Adenomatous Polyposis Coli (APC) tumour suppressor is mutated in the vast majority of human colorectal cancers (CRC) and leads to deregulated Wnt signalling. APC suppression produces adenomas in both the small intestine and colon that, in the presence of K-ras and p53 mutations, can progress to invasive carcinoma. In established tumours, APC restoration drives rapid and widespread tumour-cell differentiation and sustained regression without relapse. Tumour regression is accompanied by the re-establishment of normal crypt-villus homeostasis, such that once aberrantly proliferating cells reacquire self-renewal and multi-lineage differentiation capability. It has been shown that CRC cells can revert to functioning normal cells given appropriate signals, the Wnt pathway is a therapeutic target for treatment of CRC.

Riccio, Gennaro, et al. “WNT inhibitory activity of malus pumila miller cv annurca and malus domestica cv limoncella apple extracts on human colon-rectal cells carrying familial adenomatous polyposis mutations.” Nutrients 9.11 (2017): 1262. discloses that inhibitors of the Wnt/β-catenin pathway have been considered as potential chemopreventive agents against Familial Adenomatous Polyposis (FAP). FAP is an autosomal-dominant syndrome caused by germline mutations in the gene coding for the protein APC and leads to hyperactivation of the WNT/β-catenin signalling pathway, uncontrolled intestinal cell proliferation and formation of adenocarcinomas. It has been show that Wnt inhibitors inhibit the pathway in colon cells carrying FAP mutations with active dilutions falling in ranges close to consumer-relevant concentrations.

As such, there remains a need for new and improved treatments for preventing gastrointestinal cancers in patients who are predisposed to such cancer. For example, in patients with FAP or other disorder or disease that may lead to cancer.

There is also a need for new therapeutics for targeting and modulating the Wnt pathway of cells.

### BRIEF SUMMARY OF THE INVENTION

Provided in a first aspect of the invention is a Wnt agonist for use in preventing gastrointestinal tract cancer in a subject, wherein the subject has a genetic predisposition to gastrointestinal tract cancer.

In another aspect there is provided a method of preventing gastrointestinal tract cancer in a subject wherein the subject has a genetic predisposition to gastrointestinal tract cancer, the method comprising administering a Wnt agonist.

In certain embodiments, the cancer is a stomach, intestinal, colon or rectal cancer.

In certain embodiments, Wnt agonist inhibits the expansion of constitutively Wnt antagonist expressing cells.

In a further aspect of the invention there is provided a Wnt agonist for use in preventing or inhibiting the formation of intestinal pre-cancerous lesions or intestinal adenoma, wherein the Wnt agonist prevents or inhibits expansion of constitutively Wnt antagonist expressing cells in a subject.

In another aspect there is provided a method of preventing or inhibiting the formation of intestinal pre-cancerous lesions or intestinal adenoma in a subject, wherein the Wnt agonist prevents or inhibits expansion of constitutively Wnt antagonist expressing cells in the subject, the method the method comprising administering a Wnt agonist.

In certain embodiments, the subject has been diagnosed with Familial Adenomatous Polyposis (FAP) or attenuated FAP.

In certain embodiments, the subject has an APC mutation or a β-catenin mutation.

In certain embodiments, the APC mutation is a germline APC gene mutation.

In certain embodiments, the Wnt agonist is one or more of: a surrogate wnt; chir; R-Spondin analogue; wnt3a; Wnt 5; Wnt-6a; Norrin; CHIR99021; LiCl; BIO-Acetoxime; CHIR 98014; GSK-3 inhibitor IV; SB216763; SB415286; 5-ethyl-7,8-dimethoxy-1H-pyrrolo [3,4-c]-Isoquinoline-1,3-(2H)-dione “3F8”; 9-bromo-7,12-dihydro-indolo [3,2-d] [1 Benzazepine-6 (5H)-one “kenpaullone”; 9-bromo-7,12-dihydro-pyrido [3′, 2′: 2,3 Azepino [4,5-b]indol-6 (5H)-one “1-Azakenpaullone”; N-(3-chloro-4-methylphenyl)-5-(4-nitrophenyl)-1,3,4-oxaxe Diazole-2-amine “TC-G24”; 2-methyl-5-[3-[4-(methylsulfinyl) phenyl]-5-benzofuranyl]-1,3,4-Oxadiazole “TCS 2002”; N-[(4-methoxyphenyl) methyl]-N′-(5-nitro-2-thiazolyl) urea “AR-A014418”; 3-[5-[4-(2-hydroxy-2-methyl-1-oxopropyl)-1-piperazinyl]-2-(trifluoromethyl) phenyl]-4-(1H-indole-3-YI)-1H-pyrrole-2,5-dione “TCS 21311”; 3-[[6-(3-aminophenyl)-7H-pyrrolo [2,3-d]pyrimidin-4-yl]oxy]-phenol “TWS119”; 4-(2-Amino-4-oxo-2-imidazolin-5-ylidene)-2-bromo-4,5,6,7-tetrahydropyrrolo [2,3-c]azepine-8-one “10Z-hymenialdicine”; 2-[(3-iodophenyl) methylsulfanyl]-5-pyridin-4-yl-1,3,4-oxadiazole “GSK-3 beta inhibitor II”; 4-benzyl-2-methyl-1,2,4-thiadiazolidine-3,5-dione; FRATtide peptide; 3-amino-1H-pyrazolo [3,4-b]; Noxaline “Cdk 1/5 inhibitors”; 4-((5-bromo-2-pyridinyl) amino)-4-oxobutanoic acid “Bibikin”; Li₂CO₃; caffeine; valproic acid; ABC99; and/or LP-922056 or combinations thereof. Preferably the Wnt agonist is lithium chloride LiCl, Li₂CO₃, caffeine and/or CHIR99021 or combinations thereof.

In certain embodiments, the Wnt agonist is lithium chloride LiCl, Li₂CO₃, caffeine and/or CHIR99021 or combinations thereof.

In certain embodiments, the constitutively Wnt antagonist expressing cells decrease the number of functional wild type cells.

In certain embodiments, the Wnt agonist renders functional wild type cells insensitive to Wnt antagonist.

In certain embodiments, the Wnt agonist renders functional wild type cells insensitive to Wnt antagonist via downstream Wnt agonist mediated inhibition of GSK3β.

In certain embodiments, the Wnt agonist is administered simultaneously, separately or sequentially after administration of anti-inflammatory compound.

In certain embodiments, the anti-inflammatory compound is a NSAID; non-NSAID; selective COX-2 inhibitor; or 2-Acetoxybenzoic acid.

In certain embodiments, the Wnt agonist is administered to the subject at a sub-therapeutic amount in the bloodstream of the subject.

In certain embodiments, the Wnt agonist is LiCl or Li₂CO₃ and the subtherapeutic amount in the bloodstream of the subject of LiCl, Li₂CO₃ or a combination thereof is 0.2 mM.

In another aspect, the invention is a Wnt agonist for use in boosting fitness of wild type cells to prevent gastrointestinal tract cancer in a subject, wherein the subject has a genetic predisposition to gastrointestinal tract cancer.

In another aspect, the invention is a method of boosting fitness of wild type cells to prevent gastrointestinal tract cancer in a subject, wherein the subject has a genetic predisposition to gastrointestinal tract cancer, the method comprising administering an effective amount of a Wnt agonist to the subject.

In certain embodiments, the cancer is a stomach, intestinal, colon or rectal cancer.

In certain embodiments, the Wnt agonist inhibits the expansion of constitutively Wnt antagonist expressing cells.

In certain embodiments, the subject has been diagnosed with Familial Adenomatous Polyposis (FAP) or attenuated FAP.

In certain embodiments, wherein the subject has an APC mutation or a β-catenin mutation, optionally wherein the APC mutation is a germline APC gene mutation.

In certain embodiments, the Wnt agonist is one or more of: a surrogate wnt; chir; R-Spondin analogue; wnt3a; Wnt 5; Wnt-6a; Norrin; CHIR99021; LiCl; BIO-Acetoxime; CHIR 98014; GSK-3 inhibitor IV; SB216763; SB415286; 5-ethyl-7,8-dimethoxy-1H-pyrrolo [3,4-c]-Isoquinoline-1,3-(2H)-dione “3F8”; 9-bromo-7,12-dihydro-indolo [3,2-d][1 Benzazepine-6 (5H)-one “kenpaullone”; 9-bromo-7,12-dihydro-pyrido [3′, 2′: 2,3 Azepino [4,5-b]indol-6 (5H)-one “1-Azakenpaullone”; N-(3-chloro-4-methylphenyl)-5-(4-nitrophenyl)-1,3,4-oxaxe Diazole-2-amine “TC-G24”; 2-methyl-5-[3-[4-(methylsulfinyl) phenyl]-5-benzofuranyl]-1,3,4-Oxadiazole “TCS 2002”; N-[(4-methoxyphenyl) methyl]-N′-(5-nitro-2-thiazolyl) urea “AR-A014418”; 3-[5-[4-(2-hydroxy-2-methyl-1-oxopropyl)-1-piperazinyl]-2-(trifluoromethyl) phenyl]-4-(1H-indole-3-YI)-1H-pyrrole-2,5-dione “TCS 21311”; 3-[[6-(3-aminophenyl)-7H-pyrrolo [2,3-d]pyrimidin-4-yl]oxy]-phenol “TWS119”; 4-(2-Amino-4-oxo-2-imidazolin-5-ylidene)-2-bromo-4,5,6,7-tetrahydropyrrolo [2,3-c]azepine-8-one “10Z-hymenialdicine”; 2-[(3-iodophenyl) methylsulfanyl]-5-pyridin-4-yl-1,3,4-oxadiazole “GSK-3 beta inhibitor II”; G 4-benzyl-2-methyl-1,2,4-thiadiazolidine-3,5-dione; FRATtide peptide; 3-amino-1H-pyrazolo [3,4-b]; Noxaline “Cdk 1/5 inhibitors”; 4-((5-bromo-2-pyridinyl) amino)-4-oxobutanoic acid “Bibikin”; Li₂CO₃; caffeine; valproic acid; ABC99; and/or LP-922056; or combinations thereof. Preferably the Wnt agonist is lithium chloride LiCl, Li₂CO₃, caffeine and/or CHIR99021 or combinations thereof.

In certain embodiments, the constitutively Wnt antagonist expressing cells decrease the number of functional wild type cells.

In certain embodiments, the Wnt agonist renders functional wild type cells insensitive to Wnt antagonist, optionally via downstream Wnt agonist mediated inhibition of GSK3p.

In certain embodiments, the Wnt agonist is administered simultaneously, separately or sequentially after administration of anti-inflammatory compound, optionally wherein the anti-inflammatory compound is a NSAID; non-NSAID; selective COX-2 inhibitor; or 2-Acetoxybenzoic acid.

In certain embodiments, the Wnt agonist is administered to the subject at a sub-therapeutic amount in the bloodstream of the subject.

In certain embodiments, the Wnt agonist is LiCl or Li₂CO₃ and the subtherapeutic amount in the bloodstream of the subject of LiCl, Li₂CO₃ or a combination thereof is 0.2 mM.

In certain embodiments, boosting fitness of wild type cells limits expansion of pre-malignant clones.

In certain embodiments, the wild type cells interact with mutant cells to confer a competitive advantage to the wild type cells.

### BRIEF DESCRIPTION OF THE FIGURES

Embodiments of the invention are further described hereinafter with reference to the accompanying drawings, in which:

FIG. 1 shows Apc−/− cells actively impair outgrowth of Apc+/− organoids. a, Schematic workflow for in vitro co-cultures. b, Representative images of full wells containing WT/Apc^{+/-} co-cultures, scalebar 1 mm, and c, relative surface contribution (P=0.3771, Day 1-Day 7, two-tailed paired t-test) d, and organoid expansion in WT/Apc^{+/-} co-cultures n=4 independent experiments. e, Representative images of full wells containing Apc^{+/-}/Apc^{-/-} co-cultures, scalebar 1 mm. f, Reduction in surface contribution (P=0.0012, Day 1-Day 4), (P=0.0016, Day 1-Day 7, two-tailed paired t-test) and g, organoid expansion of Apc^{+/-} and Apc^{-/-} organoids in Apc^{+/-}/Apc^{-/-} co-culture. h, Full well images of Apc^{+/-} organoids with Apc^{+/-} or Apc^{-/-} CM at Day 7 scalebar 1 mm. Zoom panel right, 250 μm, and i, Apc^{+/-} organoid expansion in CM (P=0.0322, Day 4), (P=0.0006, Day 7). Data are mean±SD, n=3 independent experiments, analysed using two-sided Student's t-test, unless otherwise specified;

FIG. 2 shows Apc mutant cells actively impair outgrowth of WT organoids. a, Co-culture of WT/WT organoids, scalebar 1 mm, and b, relative surface contribution in WT/WT co-cultures (P=0.6905, Day 1-Day 7, two-tailed paired t-test). c, Co-culture of WT/Apc^{-/-}, scalebar 1 mm, and d, relative surface contribution in WT/Apc^{-/-} co-culture (P=0.0094, Day 1-Day 4), (P=0.0025, Day 1-Day 7, two-tailed paired t-test).e, Relative WT organoid expansion (WT vs WT co (P=0.0729, Day 4), (P=0.0002, Day 7), n=4 independent experiments). f, Relative WT cell numbers (WT vs WT co (P=0.0007, Day 4)) data is mean±SD. For FACS gating data, see Supplementary File 2. g, WT organoids incubated with WT or Apc^{-/-} CM at Day 7, scalebar 1 mm. Zoom panel right, 250 μm, and h, relative WT organoid expansion of organoids incubated with WT or Apc^{-/-} CM, (P=0.0024, Day 4), (P=0.0011, Day 7) Data are mean±SD. All data are mean±SEM, n=3 independent experiments, analysed using two-sided Student's t-test, unless otherwise specified;

FIG. 3 shows Apc mutants induce differentiation in adjacent WT cells. a, Heatmap of differentially expressed genes (DEGs) in WT organoids treated with CM (1552 DEGs). b, Phase images, c, mRNA expression and d, normalized cell type distribution of WT organoids treated with EN or ENR medium (upper panel), and treated with Apc^{-/-} CM or WT CM (lower panel), scalebar 250 μm. SC=stem cell, GC=goblet cell, PC=Paneth cell, EEC=enteroendocrine cell, E=enterocytes. e, Percentage of Lgr5-GFP^{high} cells in WT organoids treated with CM (P=0.0209). For FACS gating data, see Supplementary File 2. f, Percentage MUC2 positive cells in WT organoids incubated with CM, (P<0.0001) Boxplot is min to max, box shows 25^{th} until 75^{th} percentile, median is indicated with a line, n=25 organoids per condition, every data point is an organoid, scalebar 50 μm. g, Phase images and clonogenicity of WT organoids treated with CM (WT CM vs Apc^{-/-} CM, P=0.0041, P2), (P<0.0001, P3), data is mean±SD, scalebar 250 μm. h, Phase images and i, mRNA expression of WT human organoids treated with FAP organoid CM, scalebar 200 μm. WT CM versus FAP CM for LGR5 (P=0.0189), MUC2 (P=0.0163) and KRT20 (P=0.0607), n=4 different FAP cultures. j, Clonogenic potential of WT human organoids treated with FAP CM. WT CM versus FAP CM (P=0.0056(FAP1), 0.0014(FAP2), 0.0201(FAP3), 0.0008(FAP4)). Data are mean±SEM, n=3 independent experiments, analysed using two-sided Student's t-test, unless otherwise specified;

FIG. 4 shows Apc mutants induce differentiation in adjacent WT cells through Wnt inhibition. a-d, Signature scores for Wnt signatures (a, b) and stem cell signatures (c, d) for WT organoids treated with CM for 2 or 4 days. Signature scores were calculated by summing the standardized expression of the genes within each signature. Boxplots are min to max, box shows 25^{th} until 75^{th} percentile, median is indicated with a line, n=3 biological replicates. e-h, Effect of 10× concentrated WT or Apc^{-/-} CM on (e) WT organoid growth, scalebar 250 μm, (f) Lgr5 expression (P=0.0033, Data are mean±SEM), (g) the percentage of Lgr5-GFP^{high} cells (P=0.0371, Data are mean±SEM), and (h) the clonogenicity (P=0.0044) of WT organoids, n=4 independent experiments, i, Schematic illustration of the TOP-GFP construct. j, FACS histograms showing TOP-GFP expression in MEFs in absence (unstimulated) or presence of Wnt3a. k-m, Mean Fluorescent Intensity (MFI) of TOP-GFP upon upstream stimulation with Wnt3a (WT CM versus Apc^{-/-} CM, P<0.0001) (k), or upon downstream pathway activation with 5 mM LiCl (WT CM versus Apc^{-/-} CM, P=0.9812) (I), or 2.5 μM CHIR99021(WT CM versus Apc^{-/-} CM, P=0.8082) (m), n=4 independent experiments. Data are mean±SD, n=3 independent experiments, analysed using two-sided Student's t-test, unless otherwise specified;

FIG. 5 shows Apc mutant cells secrete Wnt antagonists. a, olcano plot for upregulated Wnt antagonists in Apc^{-/-} versus WT organoids (GSE144325, 4326 DEGs). b Expression of Notum(P=0.0067), Wif1(P=0.0006) and Dkk2(P=0.0009) in WT or Apc^{-/-} organoids. n=5, each dot represents an individual culture. c, Notum-ISH in mouse adenoma tissue, scalebar 1 mm, n=3 mice. d, Volcano plot for upregulated Wnt antagonists in human WT or APC^{KO} organoids (GSE145308, 3883 DEGs). e, Expression of NOTUM in human organoid cultures derived from healthy donors (n=4) or FAP patients, (n=5)(P=0.002). Each dot represents an individual culture. f, NOTUM-ISH in human FAP adenoma tissue, scalebar 500 μm, n=3 FAP patients g, Relative expansion and h, clonogenic capacity of WT organoids incubated with Notum, Wif1 and Dkk2 overexpression CM, or a combination of all three (P<0.0001, all conditions relative to control CM, data are mean±SD). i, Phase images and clonogenicity of WT organoids incubated with CM in the absence/presence of 5 mM LiCl (P<0.0001, One-way ANOVA, data are mean±SD), scalebar 250 μm. Data are mean±SEM, n=3 independent experiments, analysed using two-sided Student's t-test, unless otherwise specified;

FIG. 6 shows Apc mutant cells secrete Wnt antagonists. a, mRNA expression of Wnt antagonists in a time course following tamoxifen mediated recombination, n=3 technical replicates from a representative experiment performed 3 times, (P=0.0039 (Notum), P=0.0115 (Wif1), P=0.0252 (Dkk2), 72 h versus control). b, Protein levels of NOTUM and WIF1 (P<0.0001) detected in CM of WT or Apc^{-/-} organoids. c, Volcano plot for significantly upregulated Wnt antagonists in pooled normal or adenoma murine tissue (GSE65461, 2483 DEGs). d, Expression of Wnt antagonists Notum, Wif1 and Dkk2 in mouse adenoma tissue by RNA-ISH, scalebar 100 μm, n=3 mice per ISH probe e, Volcano plot for significantly upregulated Wnt antagonists in human matched normal or adenoma tissue (GSE8671, 9478 DEGs). f, NOTUM expression in FAP adenomas, scalebar 100 μm, and g, APC-mutant crypts, marked with asterisk, scalebar 100 μm. APC-mutant crypts are recognized as low-grade dysplasia by their enlarged pencillate nuclei (H&E staining, right panel), scalebar 50 μm. h, Protein levels of NOTUM in CM of WT or APC-mutant organoids (P=0.0291), Data are mean±SEM n=2 WT organoid lines, n=6 APC-mutant organoid lines. Data are mean±SD, n=3 independent experiments, analysed using two-sided Student's t-test, unless otherwise specified;

FIG. 7 shows Characterization of the role of individual Wnt antagonists. a, Schematic illustration of overexpression (OE) constructs for Notum, Wif1, and Dkk2. b, mRNA expression of Wnt antagonists in OE lines, n=3 technical replicates. c, Protein concentration in CM of OE lines, n=3 technical replicates. d, Fluorescent images, e, relative organoid expansion and f, clonogenic potential of WT organoids incubated with recombinant NOTUM(2 g/mL), WIF1(5 μg/mL) and DKK2(1 μg/mL) protein, scalebar 250 μm. P=0.0011 (rNotum), P=0.0006 (rWif1), P=0.0144 (rDkk2) and P=0.0003 (combination) all relative to the control. g, Representative image of WT/Apc^{-/-} Notum KO co-culture at day 4, scalebar 1 mm h, Relative expansion of WT organoids in co-culture with Apc^{-/-} organoids that contain CRISPR based modifications in Wnt antagonist genes Notum, Wif1, or Dkk2, n=2 single cell Apc^{-/-} KO clones per Wnt antagonist. i, Mean Fluorescent Intensity (MFI) for TOP-GFP expression in presence of Wnt3a and Apc^{-/-} KO CM (P=0.3606, one-way ANOVA, between all Apc-mutant conditions), data are mean±SEM, each dot represent a single cell Apc^{-/-} KO clone. j, Clonogenic potential of WT organoids that are incubated with a titration of Apc^{-/-} KO CM, n=2 independent experiments. k,l, Phase images (k), and clonogenicity (1) of WT human organoids incubated with recombinant NOTUM (P=0.0113 (1:200, 0.5 μg/mL), P=0.0059 (1:100, 1 μg/mL)). All data are mean±SD, n=3 independent experiments, analysed using two-sided Student's t-test, unless otherwise specified;

FIG. 8 shows Downstream activation of the Wnt pathway rescues Apc-mutant supercompetitor phenotype in vitro. a, Relative clonogenic potential of WT organoids incubated with Apc^{-/-} CM in the absence or presence of 2.5 μM CHIR (P=0.0040, P3). b, Validation of overexpression of a non-degradable variant of beta-catenin, Ctnnb^{s}, on mRNA ((P<0.0001, for Ctnnb1 vs WT and Ctnnb1 vs Apc^{-/-}), n=3 biological replicates, data are mean±SEM) and protein level. c, Fluorescent image and d, relative surface contribution of co-culture between Ctnnb^{s} (purple) and Apc^{-/-}(green) ((P=0.4604 Day 1-Day 4), (P=0.2734 Day 1-Day 7) two-tailed paired t-test), scalebar 500 μm. e, Relative LGR5 expression of human colon organoids incubated with CM in the absence or presence of LiCl (P=0.0012, FAP CM +/− LiCl). f, Relative clonogenic potential of human colon organoids incubated with WT or FAP CM in the absence or presence of LiCl (n=4 biological replicates, P<0.0001, One-way ANOVA). Data are mean±SD, n=3 independent experiments, analysed using two-sided Student's t-test, unless otherwise specified;

FIG. 9 shows LiCl neutralizes biased drift and reduces adenoma formation in Apc−/− mice. a, Recombination of the Apc-allele (Apc^{E14-16}) and co-expression of Notum, scalebar 20 μm, and b, Detection of biallelic (Notum^{pos};E14/16^{pos}) and monoallelic (Notum^{neg};E14/16^{pos}) Apc-mutants. c, d, Lgr5 expression (magenta) in WT (Notum^{neg}) cells in mixed (Notum^{neg} and Notum^{pos}) and non-mixed (Notum^{neg}) crypts in the absence (P<0.0001) (c) or presence (P=0.9587) of LiCl (d), n=75 crypts, scalebar 10 μm. Each dot represents a crypt. e, Short term in vivo experiment. f, Notum-ISH clones in control and LiCl treated mice, scalebar 20 m and g, respective clone size distributions in control or LiCl treated mice, data points indicate fractional crypt sizes per timepoint, with random x and y jitter added for visualization. Mean is indicated with a dashed line. h, i, Relative clone sizes (P=0.002, Day 21) (h) and fixation (P=0.0002, Day 21) (i) in control or LiCl treated mice, n=2 mice per condition for day 4, 7 and 10. j, Probability of fixation (P_{frx}) of Apc^{-/-} mutant cells in the presence/absence of LiCl, compared to control (neutral) drift, the line indicates the estimated mean probability for every x/y coordinate, the shaded area indicates 95% credible interval. k, Percentage Notum^{pos} crypts at day 21 (P=0.0239, n=3 mice). I, Long term in vivo experiment. m, n, Macroscopic (m) and microscopic (n) images of adenomas in distal small intestines, scalebar 2 mm) o, Total number of adenomas in control (n=9) or LiCl treated (n=12) mice (P<0.0001). All boxplots are min to max, box shows 25^{th} until 75^{th} percentile, median is indicated with a line. Data are mean±SEM, n=3 mice, analysed using two-sided Student's t-test, unless otherwise specified;

FIG. 10 shows Biallelic Apc-mutants exclusively express Wnt antagonists. a, mRNA expression of Wnt antagonists Notum, Wif1, and Dkk2 in Apc^{+/+}, Apc^{+/-} and Apc^{-/-} organoids. P<0.0001 (Notum), P=0.006 (Wif1), P=0.0004 (Dkk2). Data are mean±SEM, n=3 biological replicates, two-sided Student's t-test. b, RNA-ISH on consecutive tissue slices for detection of recombined Apc alleles (Apc^{E14-16}) and Notum in Apc^{+/+}, Apc^{+/-} and Apc^{-/-} tissues, scalebar 50 m for Apc^{+/+} and Apc^{+/-} crypt bottom images, scalebar 100 m for Apc^{-/-} adenoma. c, Expression of Notum in aging Paneth cells is not detected in young mice (upper panel, <100 days old). Notum+ Paneth cells are observed in old mice (middle panel, >800 days old), positive cells are marked with arrowheads. Notum+ Paneth cells do not interfere with Notum+/Apc^{-/-} clonal analysis and are not detected in Apc^{-/-} mice (lower panel, <100 days old), scalebar 10 μm. All RNA-ISH has been performed on n=3 mice per condition.

FIG. 11 shows Effects of LiCl on the WT mouse intestine. a, Detection of Lithium (Li+) levels in mouse serum, n=4 mice per condition. b, mRNA expression of Wnt target gene Lgr5 in isolated crypts of control (n=8) or LiCl treated (n=10) mice (P=0.0037). c, Percentage of Lgr5-GFP expressing cells in isolated crypts from control (n=11) or LiCl treated (n=12) mice, as measured by FACS, (P=0.0140). d, Fluorescent images of Lgr5-GFP+ ISCs in crypt bottoms of control or LiCl treated mice, adjacent quantification is the frequency distribution of Lgr5-GFP+ cells per half (2D) crypt, n=125 crypts per condition, each data point is a crypt bottom, scalebar 50 μm. e, Schematic illustration of in vivo treatment scheme with tamoxifen and LiCl in Lgr5-Cre^{Etr2};Rosa26^{mTmG} (WT) mice. f, Boxplot for Cre-reporter activity as measured by the percentage of induced crypts at day 4, (n=5 mice, P=0.3322) and g, Boxplots for tdTomato^{neg}/GFP^{pos} clone induction per intestinal region as measured by FACS (P=0.6335 (Prox SI), 0.5171 (Distal SI), 0.7804 (Colon). h, Fluorescent images of representative clone sizes of WT crypts of mice treated with/without LiCl, mTmGFP+ clones are visualized in white, scalebar 20 μm, and i, respective boxplots of clone size distributions of WT mice in the presence/absence of LiCl, data points indicate fractional crypt sizes per timepoint, with random x and y jitter added for visualization. Mean is indicated with a dashed line. j, Relative clone sizes (P=0.7749, Day 21) and k the relative amount of fixed clones remain unaffected by LiCl (P=0.8668, Day 21). I, No effect of LiCl on probability of replacement for WT drift. All boxplots are min to max, box shows 25^{th} until 75^{th} percentile, median is indicated with a line. Data are mean±SEM, n=3 control mice, n=2 LiCl mice, unless otherwise specified. All data are analysed using two-sided Student's t-test;

FIG. 12 shows Notum influences Lgr5 expression in adjacent crypt bottoms. a, b, Duplex RNA-ISH of Lgr5 (magenta) and Notum (blue) in crypt bottoms, scalebar 50 m (a), and relative Lgr5 expression in crypts adjacent to Notum^{pos} crypts (b) (P<0.0001, one-way ANOVA). c, d, Duplex RNA-ISH of Lgr5 (magenta) and Notum (blue) in crypt bottoms in the presence of LiCl, scalebar 50 m (c) and relative Lgr5 expression in crypts adjacent to Notum^{pos} crypts in the presence of LiCl (d) (P=0.4032, one-way ANOVA). Boxplots are min to max, box shows 25^{th} until 75^{th} percentile, median is indicated with a line, each data point represents a crypt, n=3 mice per condition;

FIG. 13 shows LiCl influences stem cell dynamics and reduces adenoma formation. a, b, The effect of LiCl on WT stem cell dynamics based on the inferred replacement probability (P_{R}) (a) or when the number of WT stem cells (N^{WT}) is determined (b). c-d, Fits of clone size distributions for the adapted stem cells model (N^{WT}) for WT and Apc^{-/-} clone dynamics in the absence (c) or presence (d) of LiCl. Each data point indicates the average clone size proportion of that particular time point, the error bars are the 95% credible interval for the proportion. Modeling is based on crypt data from n=12 mice for both the control group and the LiCl treated group. e, The amount of adenomas counted per intestinal region in the absence or presence of LiCl (P=0.0037 (proximal SI), P<0.0001 (distal SI), P=0.0077 (Colon)) n=9 (control), n=12 (LiCl). Boxplot is min to max, box shows 25^{th} until 75^{th} percentile, median is indicated with a line, each data point represents a mouse. All data are analysed using two-sided Student's t-test;

FIG. 14 shows LiCl does not influence KrasG12D stem cell dynamics. a, Schematic illustration of PCR strategy to detect wild type (Kras^{WT}) and mutant (Kras^{G12D}) alleles. b, Successful recombination of Kras^{G12D} organoids after tamoxifen administration results in loss of Lox-Stop-Lox band, which means transcription of Kras^{G12D}. c-e, Fluorescent images (c), relative Lgr5 expression (P=0.0013) (d), and clonogenicity (P=0.0007, data are mean±SD) (e) of Kras^{G12D} organoids incubated in the absence/presence of 5 mM LiCl, scalebar 250 um. n=3 independent experiments. f, Schematic illustration of in vivo treatment scheme with tamoxifen and LiCl in Lgr5-Cre^{Ert2}; Rosa26^{mTmG};Kras^{G12D} mice. g, Sorting strategy of crypts isolated from Kras^{G12D} mice 7 days after tamoxifen administration for Kras^{WT}(tdTom+) and Kras^{G12D} (GFP+) cells. h, Validation of recombination (=loss of LSL-site) of the Kras^{G12D} locus shows complete recombination in the GFP+ sorted fraction. i, Representative clone sizes of Kras^{G12D} mice treated with/without LiCl, mTmGFP+ clones are visualized in white, scalebar 20 μm, and j, respective boxplots of clone size distributions of KrasG12D mice in the presence/absence of LiCl. Boxplot is min to max, box shows 25^{th} until 75^{th} percentile, median/mean is indicated with a dashed/straight line respectively, data points indicate fractional crypt sizes per timepoint, with random x and y jitter added for visualization. Mean is indicated with a dashed line. n=2 mice per timepoint. k, Relative clone sizes (P=0.5861, Day 21, n=2 mice per timepoint) and I, the relative amount of fixed clones remain unaffected by LiCl (P=0.6718, Day 21, n=2 mice per timepoint). m, Modeling the probability of replacement (P_{R}) of Kras^{G12D} LiCl mice (versus WT LiCl mice) compared to untreated Kras^{G12D} mice (versus WT control mice). Data are mean±SEM, n=3 independent experiments, analysed using two-sided Student's t-test, unless otherwise specified. PCRs on gel (b, h) have been repeated 3 times.

FIG. 15 shows lithium carbonate (Li₂CO₃) has a similar effect on Wnt pathway activation as LiCl in the same dose range as LiCl.

FIG. 16 shows caffeine is a notum inhibitor that can rescue inhibition of Wnt singalling in vitro. This is assessed by a, b, a Wnt reporter assay and using c, d, intestinal organoids incubated with Apc-mutant conditioned medium containing Notum.

FIG. 17 shows Notum inhibitor caffeine reduces the competitive advantage of Apc-mutant cells in vivo. a, schematic illustration of the in vivo experimental set-up used in study. b, Images of intestinal crypt bottoms 21 days after loss of Apc in mice that either functioned as control or had caffeine administered in their drinking water. Apc-mutant clones were visualized by RNA-ISH for Wnt antagonist Notum c, relative clone sizes of Notum-positive crypts, and d, clone fixation in control versus caffeine treated mice over a period of 21 days.

### DETAILED DESCRIPTION

Without being bound by theory, the present inventors have shown that Apc-mutant ISCs function as bona fide supercompetitors by secreting Wnt antagonists thereby inducing differentiation of neighbouring Wild-Type (WT) ISCs. Wnt-agonists, such as LiCl prevent expansion of Apc-mutant clones and adenoma formation by rendering WT ISCs insensitive to Wnt antagonists through downstream Wnt activation. Boosting fitness of healthy cells, such as WT cells, may limit expansion of pre-malignant clones and may be a strategy to limit cancer formation in high-risk individuals or predisposed subjects. For instance, the invention is applicable for the earliest events in adenoma formation e.g., the rise of the first Apc mutant cell within a single crypt bottom.

Furthermore, without being bound by theory, the present inventors have found that pharmacological Wnt activation downstream of the ligand-receptor level, e.g. using LiCl may prevent tumour initiation, and therefore chemoprevention may be initiated at a young age. These findings provide a strategy for reducing cancer incidence in individuals predisposed or at high risk of developing gastrointestinal cancers, in particular in patients that are characterized by APC or beta-catenin mutations, for example subjects with FAP. In particular, counteracting signals from pre-malignant cells exerting a supercompetitor phenotype, may be used as a potent chemopreventive strategy in various cancers.

According to a first aspect, there is provided a Wnt agonist for use in preventing gastrointestinal tract cancer in a subject, wherein the subject has a genetic predisposition to gastrointestinal tract cancer. The present invention also covers methods of treatment of the human and animal body by therapy. Thus, any reference to a Wnt agonist for use in preventing disease also contemplates a method of preventing said disease by administering an effective amount of said Wnt agonist to a patient in need thereof. For example, the present invention may relate to a method preventing gastrointestinal tract cancer in a subject, wherein the subject has a genetic predisposition to gastrointestinal tract cancer, the method comprising administering an effective amount of the Wnt agonist to the subject.

In another aspect, there is provided a Wnt agonist for use in preventing or inhibiting the formation of intestinal pre-cancerous lesions or intestinal adenoma, wherein the Wnt agonist prevents or inhibits expansion of constitutively Wnt antagonist expressing cells in a subject.

In certain embodiments, there is provided a method of preventing gastrointestinal tract cancer in a subject, wherein the subject has a genetic predisposition to gastrointestinal tract cancer the method comprising administering an effective amount of a Wnt agonist.

In certain embodiments, there is provided a method of preventing or inhibiting the formation of intestinal pre-cancerous lesions or intestinal adenoma, comprising administering an effective amount of a Wnt agonist, wherein the Wnt agonist prevents or inhibits expansion of constitutively Wnt antagonist expressing cells in a subject.

“Wnt agonists” refers to compounds that are effective in activating the Wnt signalling pathways. The Wnt signalling pathway is a conserved pathway that regulates aspects of cell fate determination, cell migration, cell polarity, neural patterning and organogenesis during embryonic development. Wnt proteins include secreted glycoproteins and comprise a large family of nineteen proteins in humans. The Wnt signalling pathways include a group of signal transduction pathways made of proteins that pass signals from outside of a cell through cell surface receptors to the inside of the cell. Three Wnt signalling pathways have been characterized: the canonical Wnt pathway, the noncanonical planar cell polarity pathway, and the noncanonical Wnt/calcium pathway. All three Wnt signalling pathways are activated by the binding of a Wnt-protein ligand to a Frizzled family receptor, which passes the biological signal to the protein Dishevelled inside the cell. Without being bound by theory, the canonical Wnt pathway may lead to regulation of gene transcription, the noncanonical planar cell polarity pathway may regulate the cytoskeleton that is responsible for the shape of the cell, and the noncanonical Wnt/calcium pathway regulates calcium inside the cell. Wnt signalling pathway has been demonstrated to play a role in a variety of diseases, including cancer.

Wnt agonist may include any one or more of a surrogate wnt; chir; R-Spondin analogues (for example, Lgr5 agonist, such as anti-Lgr5 antibody); wnt3a; Wnt 5; Wnt-6a; Norrin; any other Wnt family protein; CHIR99021; LiCl; BIO-Acetoxime; CHIR 98014; GSK-3 inhibitor IV; SB216763; SB415286; 5-ethyl-7,8-dimethoxy-1H-pyrrolo [3,4-c]-Isoquinoline-1,3-(2H)-dione “3F8”; 9-bromo-7,12-dihydro-indolo [3,2-d][1 Benzazepine-6 (5H)-one “kenpaullone”; 9-bromo-7,12-dihydro-pyrido [3′, 2′: 2,3 Azepino [4,5-b]indol-6 (5H)-one “1-Azakenpaullone”; N-(3-chloro-4-methylphenyl)-5-(4-nitrophenyl)-1,3,4-oxaxe Diazole-2-amine “TC-G24”; 2-methyl-5-[3-[4-(methylsulfinyl) phenyl]-5-benzofuranyl]-1,3,4-Oxadiazole “TCS 2002”; N-[(4-methoxyphenyl) methyl]-N′-(5-nitro-2-thiazolyl) urea “AR-A014418”; 3-[5-[4-(2-hydroxy-2-methyl-1-oxopropyl)-1-piperazinyl]-2-(trifluoromethyl) phenyl]-4-(1H-indole-3-YI)-1H-pyrrole-2,5-dione “TCS 21311”; 3-[[6-(3-aminophenyl)-7H-pyrrolo [2,3-d]pyrimidin-4-yl]oxy]-phenol “TWS119”; 4-(2-Amino-4-oxo-2-imidazolin-5-ylidene)-2-bromo-4,5,6,7-tetrahydropyrrolo [2,3-c]azepine-8-one “10Z-hymenialdicine”; 2-[(3-iodophenyl) methylsulfanyl]-5-pyridin-4-yl-1,3,4-oxadiazole (also known as GSK-3 beta inhibitor II); 4-benzyl-2-methyl-1,2,4-thiadiazolidine-3,5-dione; FRATtide peptide; 3-amino-1H-pyrazolo [3,4-b]; Noxaline “Cdk 1/5 inhibitors”; 4-((5-bromo-2-pyridinyl) amino)-4-oxobutanoic acid “Bibikin”; LiCl; Li₂CO₃; caffeine; ABC99; LP-922056 and/or CHIR99021 or combinations thereof. A Wnt agonist may be administered alone or in combination with another Wnt agonist i.e., one or more Wnt agonists may be administered to a subject. Other Wnt agonists useful in the invention may include notum inhibitors, which inhibit the Wnt antagonist Notum thereby boosting Wnt signalling.

In certain embodiments the Wnt agonist is LiCl (lithium chloride). In certain embodiments the Wnt agonist is CHIR99021. In certain embodiments the Wnt agonist is a combination of LiCl and CHIR99021.

Gastrointestinal tract cancer refers to tumours and/or cancers of the oesophagus, stomach, small intestine, large intestine or colon as well as cancers or tumours of the annexed glands of the gastrointestinal tract, such as the liver, gallbladder biliary, the bile duct and the pancreas.

As used herein, the term “cancer” refers to cells having the capacity for autonomous growth, i.e., an abnormal state of condition characterized by rapidly proliferating cell growth. “Cancer” is meant to include all types of cancerous growths or oncogenic processes, metastatic tissues or malignantly transformed cells, tissues, or organs, irrespective of histopathologic type or stage of invasiveness. Examples of cancers include but are not limited to solid tumours and leukemias, and any conditions in which cells have become immortalized or transformed.

The cancer may be stomach, intestinal, colon or rectal cancer.

In certain embodiments, the cancer is stomach cancer. “Stomach cancer” refers to a carcinoma (malignant tumour) occurring in the stomach. For example, malignant tumours occurring in the stomach include gastric adenocarcinoma (such as diffuse type and intestinal type), lymphoma, gastric submucosal tumour, smooth myoma, leiomyosarcomas, and squamous cell carcinomas. In certain embodiments, the stomach cancer may be gastric adenocarcinoma. The symptoms of stomach cancer include discomfort in the upper abdomen, pain in the upper abdomen, indigestion, bloating, and loss of appetite, but these symptoms may be difficult to diagnose, and can usually be diagnosed by radiographic or gastroscopy among other methods.

In certain embodiments, the cancer is intestinal cancer. “Intestinal cancer” refers to cancers occurring in the intestine, including precursors of rectal cancer, colorectal cancer and intestinal cancer (e.g., adenomatous polyps).

In certain embodiments, the cancer is colon cancer. Colon cancer refers to a malignancy, for example a tumour, that arises in the large intestine (colon) or the rectum (end of the colon), and includes cancerous growths in the colon, rectum, and appendix, including adenocarcinoma. Colorectal cancer may be preceded by adenomas, neoplasms of epithelial origin which are derived from glandular tissue or exhibit clearly defined glandular structures. Colon cancer stage determination is an estimate of the invasion amount of a particular cancer. This is done for diagnostic and research purposes and to determine the optimal treatment method. The colorectal cancer stage determination system depends on the extent of local involvement, lymph node involvement and distant metastasis. Colon cancer may be diagnosed by radiographic or gastroscopy among other methods such as colonoscopy and/or CT colonography.

In certain embodiments, the caner is rectal cancer. Although colon and rectal cancer are often epidemiologically related, (i.e., colorectal cancer), rectal cancer refers to tumours that arise within 15 centimetres from the anal sphincter. Methods of staging may provide information about the location and size of a tumour in the rectum, and, if present, the size, number, and location of any metastases. In the case of rectal cancer, physical examination may be used to reveal a palpable mass and bright blood in the rectum. Diagnosis may include methods such as digital-rectal examination and/or rectovaginal exam and rigid proctoscopy, colonoscopy, pan-body computed tomography (CT) scan magnetic resonance imaging (MRI) of the abdomen and pelvis, endorectal ultrasound (ERUS), and positron emission tomography (PET) for prognostic assessment.

In certain embodiments, the cancer is a combination of two or more of stomach, intestinal, colon and/or rectal cancer.

“Prevention” or “preventing” may refer to a regimen that protects against the onset of the disease or disorder such that the clinical symptoms of the disease do not develop. Thus, “prevention” relates to administration of a therapy (e.g., administration of a therapeutic substance) to a subject before signs of the disease are detectable in the subject. The subject may be an individual at risk of developing the disease or disorder, such as an individual who has one or more risk factors known to be associated with development or onset of the disease or disorder. Such as a genetic predisposition to gastrointestinal tract cancers.

As used herein, “predisposition” refers to an increased likelihood that an individual will have a disorder. Although a subject with a predisposition does not yet have the disorder, there exists an increased propensity to the disease. A predisposed subject may have a particular genotype and/or haplotype having a higher likelihood than one not having such a genotype and/or haplotype for developing a particular disease or disorder.

Subjects with a genetic predisposition to gastrointestinal tract cancer may include subjects who are diagnosed with, suspected as suffering from, are suffering from or include specific genetic or biological markers for disorders such as Cowden syndrome, Familial Adenomatous Polyposis (FAP), attenuated FAP (AFAP), Hereditary Diffuse Gastric Cancer (HDGC), Juvenile polyposis syndrome (JPS), Lynch Syndrome, moderate risk cancer gene, MYH-associated polyposis (MAP) syndrome, Peutz-Jeghers Syndrome, POLD1 gene mutations, POLE gene mutations, and/or GREM1 gene mutations.

Subjects with Cowden syndrome have an increased risk for cancerous and non-cancerous tumours of the thyroid, breast, and endometrium, and an increased risk for colon polyps. Individuals with Cowden syndrome may have some characteristic physical features, including an above average head size (>58 cm in women and >60 cm in men) and non-cancerous bumps on their skin (trichilemmomas and papillomatous papules). Cowden syndrome is caused by mutations in the PTEN gene, and is inherited in an autosomal dominant fashion. This means that children, brothers, sisters, and parents of subjects with Cowden syndrome have a 50% risk to have Cowden syndrome. Subjects with a mutation for Cowden syndrome may develop one cancer, more than one cancer, or none at all.

HDGC accounts for less than 1-3% of all gastric cancer. Diffuse Gastric cancer is a specific type of invasive stomach cancer that thickens the wall of the stomach wall without forming a distinct tumour. Diffuse gastric cancer is also called signet ring carcinoma or isolated cell-type carcinoma. Women with HDGC have a significantly increased risk to develop lobular breast cancer. Individuals with HDGC also have an increased risk for certain other types of breast cancer, as well as colon cancer and pancreatic cancer. HDGC is diagnosed based on a patient's personal and family history of cancer. About 30-50% (⅓ to ½) of people with HDGC have a mutation in the CDH1 gene that can be identified by blood testing. In some hereditary families a genetic mutation may not be detected by the current technology, therefore close relatives will still need to be treated as high risk. Any child, brother, sister, or parent of a subject who has CDH1 mutation has a 50% chance of also having the mutation.

MAP syndrome is a form of inherited colorectal cancer. Individuals with MAP syndrome develop multiple polyps (adenomas) in the colon. Individuals with MAP syndrome usually have between 15 and 100 polyps, but MAP syndrome can occur in subjects with less than 15 polyps or greater than 100 polyps. Aside from colon polyps, individuals with MAP syndrome can develop tumours in the upper gastrointestinal system, CHRPE (multiple areas of pigmentation in the retina of the eye), osteomas (benign bone tumours) of the jaw, impacted teeth, extra teeth, and benign tumours of the hair follicle. If left untreated, the polyps in the colon may develop in to cancer. Individuals with MAP syndrome caused by two mutations in the MYH gene have up to an 80% risk to develop colon cancer in their lifetime and a 5% risk to develop cancer in the duodenum. Individuals who have one mutation in the MYH gene also seem to have an increased risk for colon cancer. MAP syndrome is caused by mutations in the MYH gene. MAP syndrome is inherited in an autosomal recessive fashion, meaning that a person must inherit a mutation in the MYH gene from both of their parents to have MAP syndrome. Brothers and sisters of a person with MAP have a 25% (1 in 4) risk to inherit MAP syndrome, a 50% (1 in 2) risk to have one MYH mutation, and a 25% (1 in 4) chance that they will not have a MYH mutation. Approximately 1-2% of the population has a MYH mutation, individuals with one MYH mutation have an increased risk for having a child with MAP syndrome, and their spouse should be offered testing to see if they also have a MYH mutation.

Moderate risk cancer gene relates to subjects with CHEK2 gene mutations which leads to an increased risk for cancers of the breast, colon, prostate, and possibly thyroid and kidney. Exact lifetime cancer risks for individuals with mutations in this gene are currently not well understood, since CHEK2 mutations seem to work in conjunction with other cancer susceptibility genes to modify risk. Mutations in the CHEK2 gene are inherited in an autosomal dominant fashion. This means that children, brothers, sisters, and parents of individuals with a CHEK2 mutation have a 50% chance of having the mutation as well. Individuals with a CHEK2 mutation may develop one cancer, more than one cancer, or none at all.

Lynch syndrome is one of the most common causes of inherited colon cancer, and accounts for 3-5% of all colon cancers. Families with Lynch syndrome often have multiple family members with colon, uterine or other cancers, typically diagnosed before age 50. Lynch syndrome is caused by mutations in one or more of five different genes, and the specific cancer risks and management recommendations depend on the gene(s). Individuals with Lynch syndrome have up to an 80% risk to develop colon cancer. Women with Lynch syndrome have a 40-60% risk for uterine cancer and a 10-12% risk for ovarian cancer. Men and women with Lynch syndrome also have an increased risk for stomach, small intestine, biliary tract, urinary tract, pancreatic and brain cancers. Lynch syndrome is inherited in an autosomal dominant fashion, and is caused by mutations in any one or more of the following genes: MLH1, MSH2, MSH6, PMS2 and EPCAM. Children, brothers, sisters, and parents of an individual with Lynch syndrome have a 50% risk to have a mutation. Individuals with Lynch syndrome may develop one cancer, more than one cancer, or none at all.

JPS may lead to gastrointestinal tract juvenile-type hamartomatous polyps (stomach, small intestine, colon, and rectum); as well as cancers of the colon, stomach, upper GI, and pancreas. JPS may be identified by mutations in the genes BMPR1A and SMAD4.

FAP is a condition that accounts for about 1% of cases of colorectal cancer. People with FAP typically develop hundreds to thousands of polyps (adenomas) in their colon and rectum by age 30-40. Polyps may also develop in the stomach and small intestine. Individuals with FAP can develop non-cancerous cysts on the skin (epidermoid cysts), especially on the scalp. Besides having an increased risk for colon polyps and cysts, individuals with FAP are also more likely to develop sebaceous cysts, osteomas (benign bone tumours) of the jaw, impacted teeth, extra teeth, CHRPE (multiple areas of pigmentation in the retina in the eye) and desmoid disease. Some individuals may have a milder form of FAP, called attenuated FAP (AFAP), and develop an average of 20 polyps at a later age. If left untreated, the polyps in the colon and rectum may develop in to cancer, usually before age 50. Individuals with FAP also have an increased risk for stomach cancer, papillary thyroid cancer, periampullary carcinoma, hepatoblastoma (in childhood), and brain tumours. FAP is caused by mutations in the Adenomatous Polyposis Coli (APC) gene. Approximately ⅓ of people with FAP do not have a family history of the disease, and thus have a new mutation. FAP is inherited in a dominant fashion. Children of a person with an APC mutation have a 50% risk to inherit the mutation. Almost all subjects who have an APC mutation develop FAP.

In certain embodiments, the subject has been diagnosed with Familial Adenomatous Polyposis (FAP) or attenuated FAP.

In certain embodiments, the subject suffers from or is predisposed to precancerous conditions. “Pre-cancerous condition” is characterized as a slowly growing colony that, if unchecked, has the potential to give rise to cancer (e.g., neoplastic tumours). Such conditions are characterized, for example, by the presence of an abnormal microanatomical structure or structures such as, for example, polyps in the colon or bell-shaped nuclei within an adult tissue sample.

In certain embodiments, the subject is predisposed to gastrointestinal polyps. “Polyp” refers to a growth of excess tissue that forms in the gastrointestinal tract for example on the lining of the, small intestine, large intestine (colon), or rectum. The terms “polyp” and “lesion” may be used interchangeably throughout.

The most common types of colon and rectal polyps include adenomatous (tubular adenoma); villous adenoma (Tubulovillous Adenoma); Hyperplastic; Serrated; and Inflammatory.

Adenomatous (tubular adenoma) polyps refer to raised protrusions of colonic mucosa, i.e., polyps formed by glandular tissue. Adenomas are usually considered precancerous and can transform into malignant structures. Adenomatous polyps are commonly asymptomatic. These growths may be found on screening colonoscopies. If symptomatic, the most frequent symptom is haematochezia, i.e., painless bright or dark red blood per rectum on wiping or with bowel movements mixed with stools or dripping. Occasionally subjects might have a history of alterations in bowel movements (either diarrhoea or constipation), weight loss, loss of appetite, abdominal pain, symptoms of partial bowel occlusion, or iron deficiency anaemia due to bleeding.

Villous adenoma (Tubulovillous Adenoma) polyps may be characterized by more than 75% villous features, where villous refers to finger-like or leaf-like epithelial projections. Tubulovillous adenomas have between 25% and 75% villous features. Less than 25% villous features indicate a tubular adenoma. Adenomas with villous features may be associated with an increase in development of more advanced neoplasia or dysplasia compared to other types of adenomas. Villous adenomas tend to occur more frequently in the rectosigmoid area but can occur elsewhere in the colon. Villous adenomas account for 5% to 15% of all adenomas.

Hyperplastic polyps refer to a type of serrated polyp. They are often found in the distal colon and rectum. They have no malignant potential, which means that they are no more likely than normal tissue to eventually become a cancer.

Serrated polyps include sessile serrated lesions (SSL) which include a premalignant flat (or sessile) lesion of the colon, predominantly seen in the cecum and ascending colon.

Inflammatory polyps refers polyps that occur in people who have inflammatory bowel disease (IBD). These types of polyps are also known as pseudo polyps because they may not be considered to be true polyps, but rather develop as a reaction to chronic inflammation in the colon.

In certain embodiments, the subject has a predisposition for a gastrointestinal inflammatory disorder such as IBD. The term “inflammatory bowel disease” or “IBD” refers to, for example, Crohn's disease (CD), ulcerative colitis (UC), indeterminate colitis (IC), and IBD where CD or UC is not definitive, “non-deterministic” gastrointestinal disorders.

In certain embodiments, the subject has been diagnosed with Familial Adenomatous Polyposis (FAP) or attenuated FAP.

In certain embodiments, the subject has an APC mutation or a β-catenin mutation, optionally wherein the APC mutation is a germline APC gene mutation.

Mutations in the β-catenin gene (CTNNB1) have been implicated in the pathogenesis of some cancers. High incidences of CTNNB1 mutations have been detected in endometrial, liver, and colorectal cancers. Elevated frequencies of missense mutations have been found in the exon 3 of CTNNB1, which is responsible for encoding the regulatory amino acids at the N-terminal region of the protein. In the case of metastatic colorectal cancers, in frame deletions have been found in the region spanning exon 3. β-Catenin is an important co-activator downstream of the Wnt signalling pathway. β-Catenin is a multitasking protein involved in transcription and cell adhesion. In particular, β-catenin is an important co-activator of Wnt target genes, such as cyclin D1 and c-myc. Wnt/β-catenin signalling plays an important role in the tumorigenesis of CRC. In particular, alteration of APC, has been found in approximately 70% of CRC patients. Several studies reported that R-catenin has oncogenic activity in CRC cells. Beta catenin is identified under the UniProtKB ascension number P35222.

Examples of effects of APC gene mutations can be found in “Aoki, Koji, and Makoto M. Taketo. “Adenomatous polyposis coli (APC): a multi-functional tumor suppressor gene.” Journal of cell science 120.19 (2007): 3327-3335”. APC gene product is a 312 kDa protein that has multiple domains, through which it binds to various proteins, including β-catenin, axin, CtBP, Asefs, IQGAP1, EB1 and microtubules. Studies using mutant mice and cultured cells have demonstrated that APC suppresses canonical Wnt signalling, which is involved in tumorigenesis, development and homeostasis of a variety of cell types, such as epithelial and lymphoid cells. Further studies have suggested that APC plays roles in several other fundamental cellular processes. These include cell adhesion and migration, organization of the actin and microtubule networks, spindle formation and chromosome segregation. Deregulation of these processes caused by mutations in APC is implicated in the initiation and expansion of colon cancer. The APC protein is identified under the UniProtKB ascension number P25054.

Mutation refers to one or more gene changes (mutations), such as one or more deletions, substitution, polymorphisms, insertions, duplications, nonsense mutations, missense mutations, frameshift mutations and/or repeat expansions, or combinations thereof. Germline mutations refer to mutations in a subject's reproductive cell (egg or sperm) that becomes incorporated into the DNA of every cell in the body of the offspring. Germline mutations are passed on from parents to offspring.

Mutations in the APC gene can include: R99W; S1711; R414C; S722G; S784T; E911G; P1176L; A1184P; T1292M; T1313A; R1348W; S2621C; and/or L2839F or any combination thereof with reference to the APC protein is identified under the UniProtKB ascension number P25054.

Loss or reduction of functional APC protein in cells, for example via mutations in the APC gene, may be lead to upregulation of one or more Wnt antagonists, for example, Palmitoleoyl-Protein Carboxylesterase (Notum), Wnt inhibitory factor 1 (Wif1) and/or Dickkopf-related protein 2 (Dkk2).

In certain embodiments, cells comprising one or more APC mutations constitutively express Wnt antagonists. In certain embodiments, cells comprising one or more APC mutations are insensitive to Wnt modulation at the receptor level. In certain embodiments, cells comprising one or more APC mutations actively supress wild-type cell expansion and/or function. In certain embodiments, cells comprising one or more APC mutations act as supercompetitor cells in respect of wild-type cells.

In certain embodiments, cells comprising one or more APC mutations constitutively express Palmitoleoyl-Protein Carboxylesterase (Notum). In certain embodiments, cells comprising one or more APC mutations constitutively express Wnt inhibitory factor 1 (Wif1).

In certain embodiments, cells comprising one or more APC mutations constitutively express Dickkopf-related protein 2 (Dkk2).

Cells with one or more APC gene mutations may also cause reduced stem cell functionality, reduced expansion rates, growth suppression, increase differentiation, reduce clonogenicity and/or decrease stemness of wild-type cells. For example, via the upregulated Wnt antagonists.

In certain embodiments, the Wnt agonist renders wild type cells insensitive to Wnt antagonists. That is to say that Wnt antagonists do not have the effects on wild-type cells such as reduced stem cell functionality, reduced expansion rates, growth suppression, increase differentiation, reduce clonogenicity and/or decrease stemness. In certain embodiments, the Wnt agonist leads to an upregulation of Notum in wild-type cells.

In certain embodiments, wild-type cells are intestinal stem cells (ISCs). In certain embodiments, the cells comprising an APC gene mutation are ISC cells.

The intestinal epithelial villus/crypt structure, its surrounding pericryptal fibroblasts, and mesenchyme form an anatomical unit that generates four cell lineages, namely absorptive enterocytes and goblet, enteroendocrine, and Paneth cells of the secretory lineage. The crypt is a contiguous pocket of epithelial cells at the base of the villus, in which intestinal stem cells (ISCs) are periodically activated to produce progenitor or transit amplifying (TA) cells which are committed to produce mature cell lineages. In normal conditions, newly formed TA cells reside within the crypts for 2-3 days and undergo up to six rounds of cell division. When these newly divided cells reach the crypt-villus junction, they rapidly differentiate into each of the four terminally differentiated cell types. The crypt is mainly occupied by undifferentiated cells; but differentiated Paneth cells that secrete antibacterial peptides into the crypt are also located at the base of the crypt area and escape their upward migration.

In certain embodiments, the wild-type and/or cells comprising one or more APC mutations are located in a crypt.

In certain embodiments, the Wnt agonist is administered simultaneously, separately or sequentially after administration of anti-inflammatory compound. “Anti-inflammatory agent” refers to an agent or combination of agents that may be used to relieve swelling, pain, and other symptoms of inflammation.

In certain embodiments, the anti-inflammatory compound is a NSAID; non-NSAID; selective COX-2 inhibitor; or 2-Acetoxybenzoic acid.

Examples of NSAIDs include Propionic acid drugs such as Fenoprofen calcium (Nalfon®), Flurbiprofen (Ansaid®), Suprofen. Benoxaprofen, Ibuprofen (prescription Motrin®), Ibuprofen (200 mg. over the counter Nuprin, Motrin 1B®), Ketoprofen (Orduis, Oruvall®), Naproxen (Naprosyn®), Naproxen sodium (Aleve, Anaprox, Aflaxen®), Oxaprozin (Daypro®), or the like; Acetic acid drug such as Diclofenac sodium (Voltaren®), Diclofenac potassium (Cataflam®), Etodolac (Lodine®), Indomethacin (Indocin®), Ketorolac tromethamine (Acular, Toradol® intramuscular), Ketorolac (oral Toradol®), or the like; Ketone drugs such as Nabumetone (Relafen®), Sulindac (Clinoril®), Tolmetin sodium (Tolectin®). or the like; Fenamate drugs such as Meclofenamate sodium (Meclomen®), Mefenamic acid (Ponstel®), or the like; Oxicam drugs such as Piroxicam (Dolibid®), or the like; Salicylic acid drugs such as Diflunisal (Feldene®), Aspirin, or the like; Pyrazolin acid drugs such as Oxyphenbutazone (Tandearil®), Phenylbutazone (Butazolidin®), or the like; acetaminophen (Tylenol®), or the like; or mixtures or combinations thereof.

COX-2 inhibitors include Celebrex, Vioxx, or the like, or mixtures or combinations thereof.

“Subject”, “individual” or “patient” are used interchangeably and refer to a human or any non-human animal (e.g., mouse, rat, rabbit, dog, cat, cattle, swine, sheep, horse or primate).

In certain embodiments, the subject is a human.

The term “effective amount” or “therapeutically effective amount” refers to an amount of a compound effective to treat, prevent or inhibit a disease or disorder in a subject. An effective amount can be administered in one or more administrations, applications or dosages and is not intended to be limited to a particular formulation or administration route.

As used herein, the term “administered” or “administering” refers to any method of providing a Wnt agonist, for example in a composition to a subject such that the Wnt agonist has its intended effect on the patient. For example, one method of administering is by an indirect mechanism using a medical device such as, but not limited to a catheter, applicator gun, syringe etc. A second exemplary method of administering is by a direct mechanism such as, local tissue administration (i.e., for example, extravascular placement), oral ingestion, transdermal patch, topical, inhalation, suppository.

An effective amount may be determined by titration to optimize safety and effectiveness. Lower than expected dosages may be administered first to a subject, and dosages may then be titrated upward until a therapeutically effective and safe concentration or amount (or a potentially unsafe concentration or amount) is reached.

Appropriate dosage amounts for a Wnt agonist may be determined or predicted from empirical evidence. Specific dosages may vary according to numerous factors and may be initially determined on the basis of in vitro, cell culture, and/or animal in vivo studies. Dosages or concentrations tested in vitro for a Wnt agonist according to some embodiments of the present invention may provide useful guidance in determining therapeutically effective and appropriate amounts for in vivo administration. For example, a therapeutically effective dose of a Wnt agonist may be estimated initially from a cell culture assay, for example, by measuring proliferation, growth, and/or survival of cultured cells or by the formation of vessels in culture in response to the Wnt agonist depending on the intended therapeutic application. Such values may be used, for example, to translate into appropriate amounts for use in animal testing or for clinical trials in humans. Determining an appropriate dosage for a Wnt agonist according to embodiments of the present invention may be discerned from any or all information or data available from any assay or experiment performed.

Animal testing of predicted dosages may provide additional indication of proper dosages for other types of animals, including humans.

In most cases, an appropriate dosage amount may be a balance of factors including efficacy and safety. Factors considered in determining a dosage that is therapeutically effective and safe for an individual, subject, or patient in clinical settings will depend on many factors including the mode/route of administration, timing of administration, rate of excretion, target site, disease or physiological state, medical history, age, sex, physical characteristics, other medications, etc. This list of factors is illustrative and not exhaustive, and may include any or all factors which might be considered by a skilled scientist, veterinarian, or physician (as the case may be) in determining an appropriate treatment. A specific dosage amount of a Wnt agonist administered to an individual, subject, or patient may be in a range equivalent to dosages used for other currently-used a Wnt agonists, adjusted for the altered activity, thermostability, or functional half-life of the particular a Wnt agonist.

In certain embodiments, the Wnt agonist is part of a composition. In certain embodiments, the composition is a pharmaceutical composition. The term “pharmaceutical composition” refers to a mixture of one or more of Wnt agonist and/or anti-inflammatory agents, pharmaceutically acceptable salts, solvates, hydrates or prodrugs thereof and other chemical components (such as a pharmaceutically acceptable carrier). The pharmaceutical composition is used to facilitate the process of the administration of one or more of Wnt agonist and/or anti-inflammatory agents to a subject. In addition to the pharmaceutically acceptable carriers, the pharmaceutical compositions may also include pharmaceutically commonly used adjuvants, such as anti-bacterial agent, antifungal agent, antimicrobial agent, preservative, colour matching agent, solubilizer, thickener, surfactant, complexing agent, protein, amino acid, fat, carbohydrate, vitamin, mineral, trace element, sweetener, pigment, flavour and/or a combination thereof.

The term “pharmaceutically acceptable carrier” refers to a non-active ingredient in the pharmaceutical composition, which can be any one or more of: calcium carbonate, calcium phosphate, various carbohydrates (such as lactose, mannitol, etc.), starch, cyclodextrin, magnesium stearate, cellulose, magnesium carbonate, acrylic polymer, methacrylic polymer, gel, water, polyethylene glycol, propylene glycol, ethylene glycol, castor oil, hydrogenated castor oil, polyethoxylated hydrogenated castor oil, sesame oil, corn oil, peanut oil and the like.

In certain embodiments, the Wnt agonist is for use in boosting fitness of healthy cells such as wild type cells to prevent gastrointestinal tract cancer in a subject, wherein the subject has a genetic predisposition to gastrointestinal tract cancer. Boosting the fitness of healthy cells such as wild type cells may involve limiting the expansion of pre-malignant clones. Boosting the fitness of healthy cells such as wild type cells may mean maintaining healthy/wild type cells in a healthy/wild type state. Boosting the fitness of wild type cells may involve modulation of the local environment of a cancer which may involve interactions between healthy/wild type cells and non-healthy/mutant cells. Boosting the fitness of healthy cells such as wild type cells may confer a competitive advantage to wild type cells over mutant cells. Boosting the fitness of healthy cells such as wild type cells prevents the transformation of pre-malignant (precancerous cells or early stage cancerous cells) into malignant cells.

In certain embodiments, the Wnt agonist is administered to a subject at a subtherapeutic amount in the bloodstream of the subject. A subtherapeutic amount of Wnt agonist may be an amount that is less than the amount currently used in the field and required for treating diseases, disorders, or conditions e.g., it is an amount that is currently used in the field which does not achieve a therapeutic effect in the treatment of particular diseases, disorders, or conditions or is not optimal at treating particular diseases, disorders or conditions. A subtherapeutic amount of Wnt agonist may be about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% lower than an amount of Wnt agonist used to achieve a therapeutic effect in known methods of treating diseases, disorders, or conditions. sub-therapeutic amount of Wnt agonist may be about 10%-20%, 20-30%, 30-40%, 40-50%, 60-70%, 80-90%, 90-99.9% lower than an amount of Wnt agonist used to achieve a therapeutic effect in known methods of treating diseases, disorders, or conditions. For example, LiCl is known to treat bipolar disorder and concentrations used to treat bipolar disease is known in the art. Thus, a subtherapeutic level of LiCl may be less than the amount used to treat bipolar disorder. Preferably, a sub-therapeutic amount in the bloodstream of a subject is a concentration of 0.2 mM for LiCl or Li₂CO₃ or a combination thereof. Preferably, a sub-therapeutic amount in the bloodstream of a subject is a concentration of 0.4 mM for LiCl or Li₂CO₃ or a combination thereof. Preferably, the concentration of Wnt agonist in the bloodstream is 0.1 mM-0.2 mM, More preferably, the concentration of Wnt agonist in the bloodstream is 0.2 mM-0.4 mM.

The inventors set out to study how Apc-mutant clones exert their competitive advantage over WT ISCs with the aim of identifying signals amenable to pharmacological manipulation. This was achieved by using the combined strengths of in vitro organoid cultures and detailed analyses of in vivo clonal dynamics.

Aspects of the present invention will now be illustrated by way of example only and with reference to the following experimentation.

### EXAMPLES

### Example 1—Apc-Mutant Cells Actively Outcompete Wild Type Cells

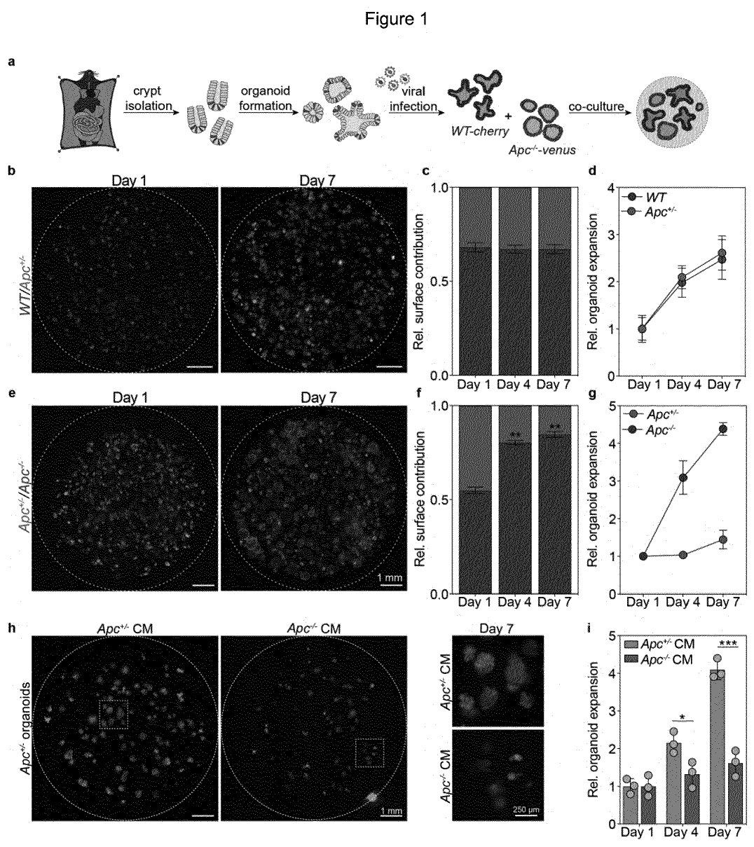

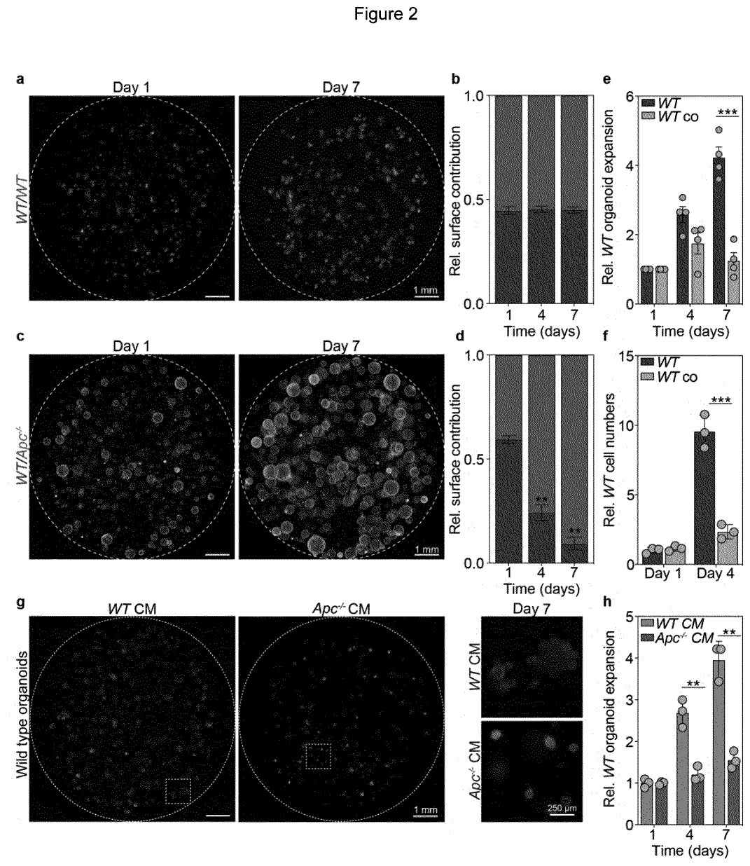

A co-culture system of WT and Apc^{-/-} organoids transduced with distinct fluorescent labels was established (FIG. 1a). Whilst the relative surface contribution in WT/WT co-cultures remained constant over time (FIGS. 2a and b), Apc^{-/-} organoids rapidly dominated the co-cultures with WT organoids (FIGS. 2c and d) mimicking previous observations in vivo^{3,7}. This was not simply caused by different proliferation rates of Apc^{-/-} and WT organoids, instead the WT organoids displayed reduced expansion rates when co-cultured with Apc^{-/-} cells, demonstrating that Apc-mutant cells actively suppress growth of WT organoids as determined by surface expansion and cell numbers (FIGS. 2e and f). The growth suppressive effect is mediated by secreted factors as conditioned medium (CM) from Apc^{-/-} organoids, supplemented with fresh growth factors, had a comparable effect (FIGS. 1g, h). Typically, Apc^{-/-} cells arise in a crypt containing Apc^{+/-} cells following loss of heterozygosity^{8,9}. While Apc^{+/-} cells displayed similar expansion rates as Apc^{+/+} organoids (FIG. 1b-d), it was observed that Apc^{-/-} cells also had a growth-reducing effect on Apc^{+/-} organoids in co-culture (FIG. 1e-g). In agreement, Apc^{-/-} CM also reduced growth of Apc^{+/-} organoids (FIGS. 1h, i). These results indicate that intestinal Apc^{-/-} cells act as supercompetitors, as they do not passively outcompete their WT and Apc^{+/-} counterparts but actively subjugate growth of neighbouring cells.

### Example 2—Apc-Mutant Cells Induce Differentiation in Wild Type Organoids

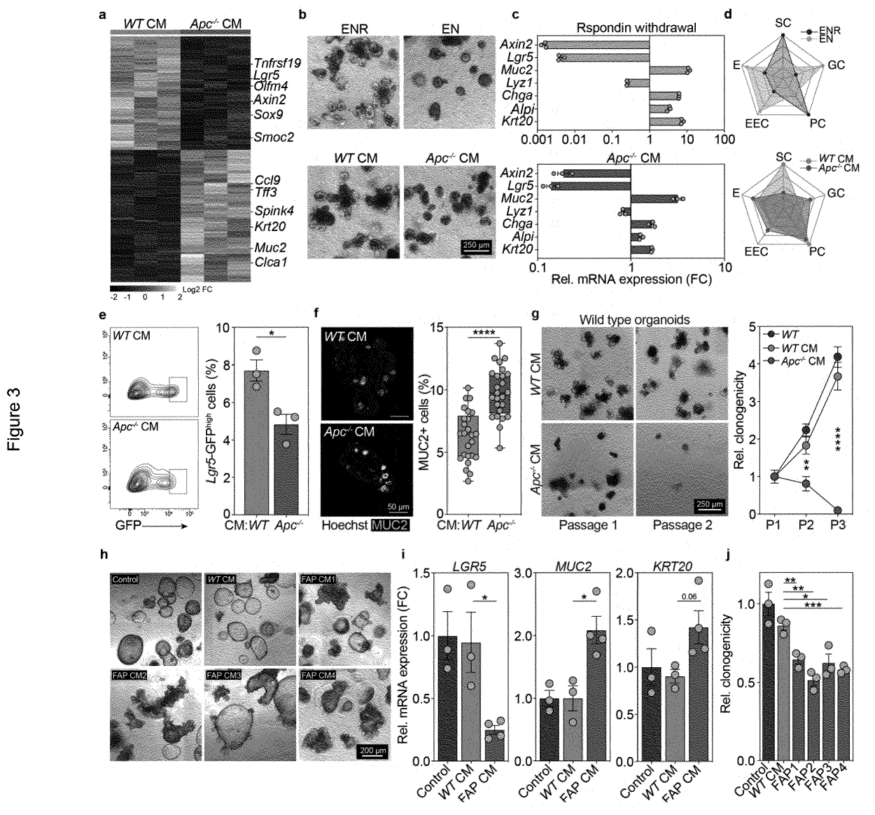

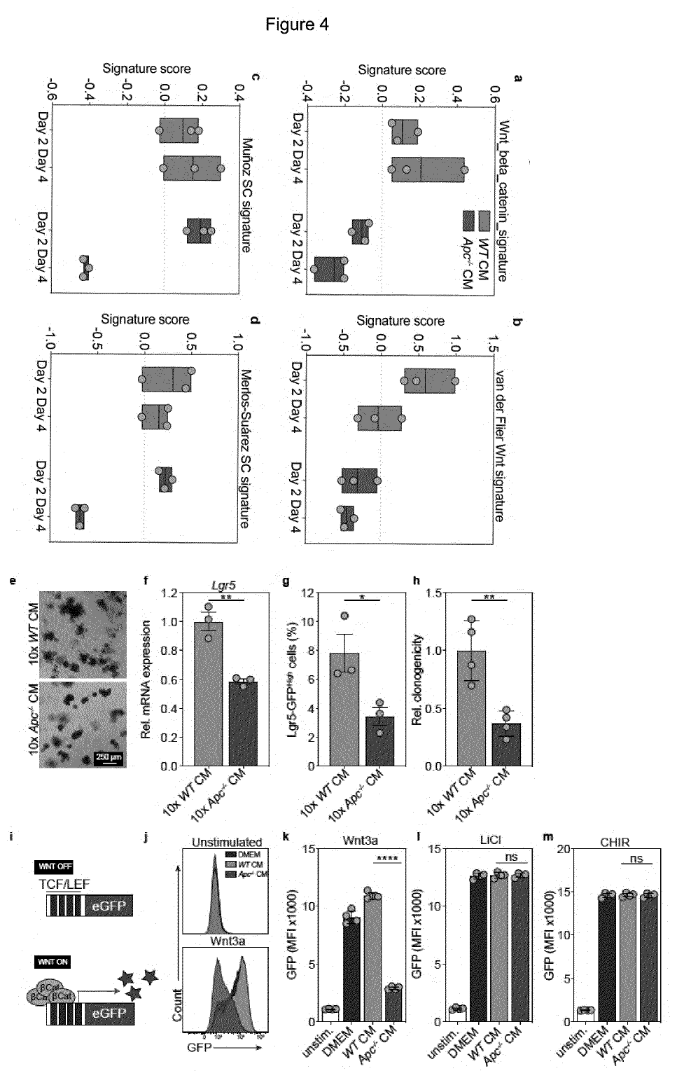

To further understand the suppressive influence mediated by Apc-mutant cells on their WT counterpart, transcriptome analysis on WT organoids treated with either WT or Apc^{-/-} CM was performed (FIG. 3a). WT organoids incubated with Apc^{-/-} CM displayed features of decreased sternness and increased differentiation as evidenced by reduced expression of Wnt and ISC signatures (FIG. 4a-d). These changes mimic the differentiation pattern observed in organoids following R-spondin withdrawal (FIGS. 3c and d)¹⁰. The preceding transcriptome-based analyses were confirmed by the fact that WT organoids established from Lgr5-GFP mice that were exposed to Apc^{-/-} CM, displayed reduced numbers of Lgr5-GFP+ cells (FIG. 3e). Furthermore, increased MUC2-positive goblet cell numbers in organoids treated with Apc^{-/-} CM were confirmed (FIG. 3f). In addition, factors produced by Apc^{-/-} cells reduced stem cell functionality, as serial passaging of WT organoids exposed to Apc^{-/-} CM demonstrated markedly reduced clonogenic capacity (FIG. 3g). These findings were corroborated in a human context using organoids established from polypectomies of four individuals with genetically confirmed familial adenomatous polyposis (FAP). CM from APC^{-/-} organoids from FAP individuals reduced LGR5 expression, increased differentiation markers MUC2 and KRT20, and reduced clonogenicity in WT human organoids (FIG. 3h-j),Together, these data reveal that Apc/APC-mutant murine and human cells secrete factors that actively suppress outgrowth and clonogenicity of WT organoids by promoting differentiation and reducing stem cell numbers.

To determine the nature of the cellular mediators responsible for these observations, the possibility that consumption of metabolites and growth factors by Apc^{-/-} organoids was involved was ruled out, as supplementing fresh medium with concentrated CM exerted similar effects (FIG. 4e-h). Given the similarity in phenotype to R-spondin withdrawal, and the decrease in Wnt pathway signatures, it was considered that CM from Apc^{-/-} organoids supressed Wnt activity in WT cells. Apc^{-/-} CM significantly reduced recombinant Wnt3a mediated Wnt activation in mouse embryonic fibroblasts (MEFs) that carried a TOP-GFP reporter (FIG. 4i-k). Downstream activation of the Wnt pathway by GSK3p inhibitors lithium chloride (LiCl) or CHIR99021 (CHIR) completely abrogated this effect, indicating that pathway inhibition occurs upstream at the ligand/receptor level (FIG. 4l-m).

### Example 3—Apc-Mutant Cells Secrete Wnt Antagonists

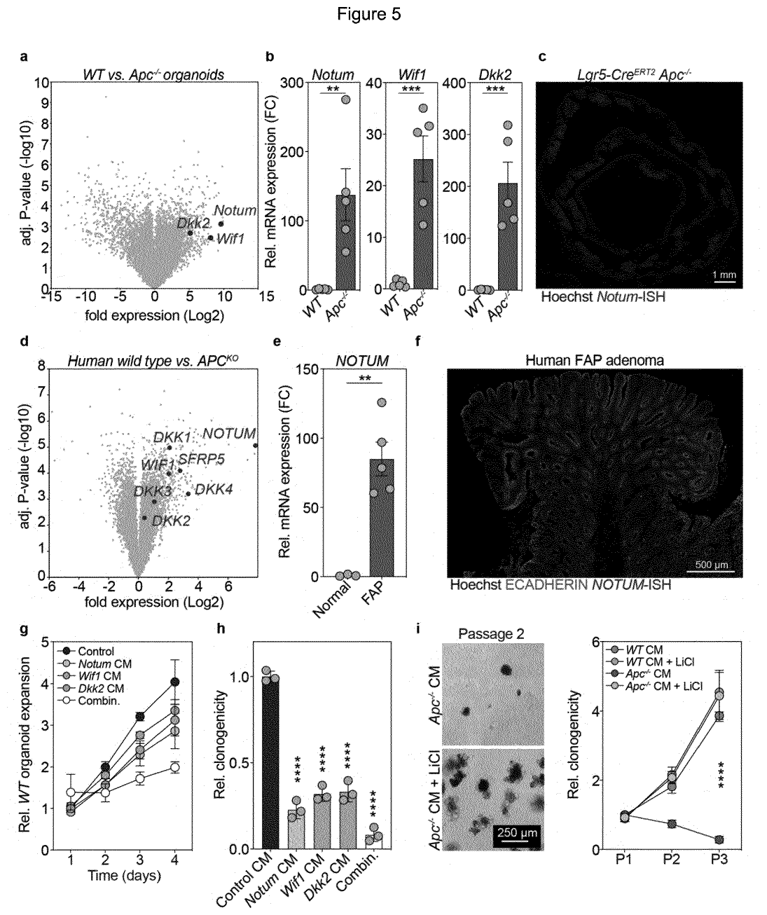

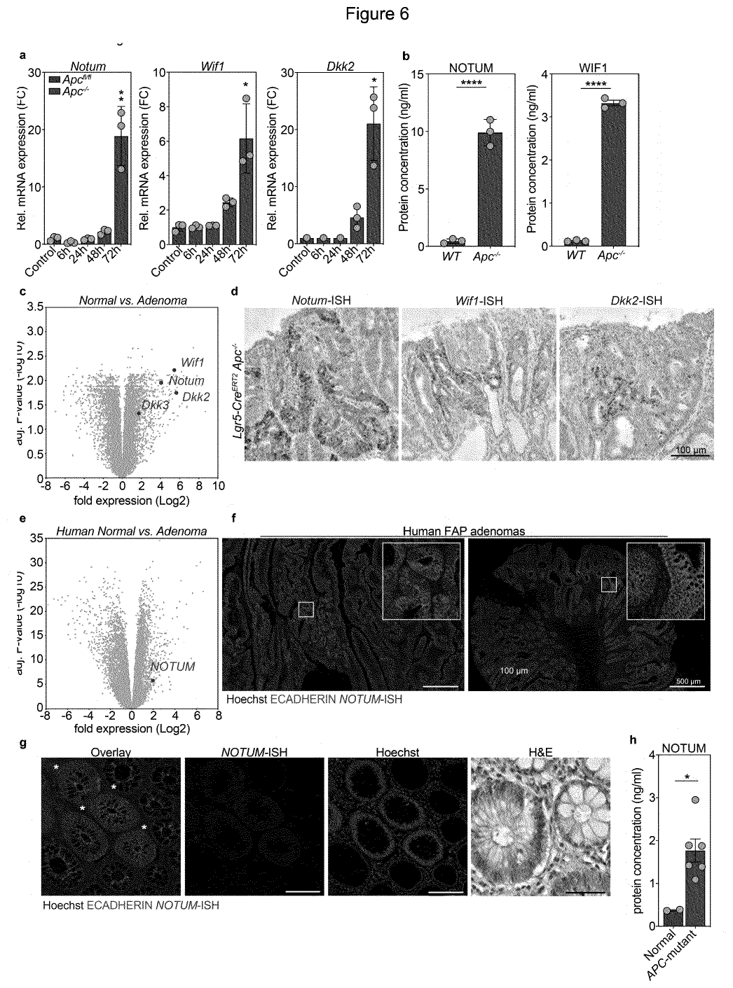

Transcriptome analysis of murine WT and Apc^{-/-} organoids revealed that loss of Apc is accompanied by a marked upregulation of several Wnt antagonists, in particular Palmitoleoyl-Protein Carboxylesterase (Notum), Wnt inhibitory factor 1 (Wif1) and Dickkopf-related protein 2 (Dkk2) (FIGS. 5a, b). In a time-course experiment it was confirmed that rapid upregulation of these Wnt antagonists following Apc inactivation in organoid cultures as well as their production in CM (FIGS. 6a, b). In agreement, it was identified that upregulation of the same antagonists in murine adenomatous tissue in vivo (FIG. 5c, FIGS. 6c, d). Importantly, a series of partially similar Wnt antagonists were also found upregulated in human APC-deficient organoids (FIG. 5d). In particular NOTUM was also highly upregulated in human FAP derived APC-deficient organoids and adenomas (FIGS. 5e, f, FIGS. 6e-h).

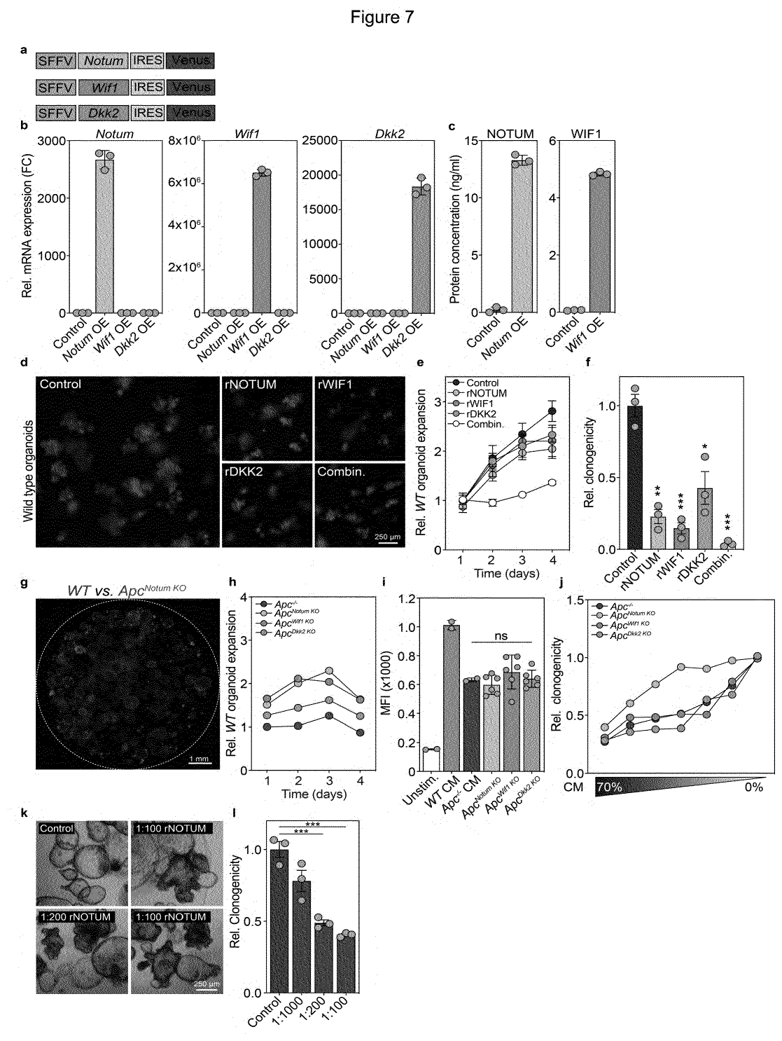

Without being bound by theory the findings suggest that persistent Wnt pathway signalling results in activation of a potent negative feedback loop, involving upregulation of Wnt antagonists, that in physiological circumstances is likely to regulate Wnt levels. Of note, whereas Apc^{-/-} cells are insensitive to Wnt modulation at the receptor level, Apc-proficient cells are not, resulting in loss of stem cell features. To determine which antagonists are responsible for the observed effect, WT organoids were treated with CM generated from cells overexpressing Notum, Wif1 or Dkk2, or with the recombinant variants (FIGS. 5g, h, FIGS. 7a-f). It was found that all three antagonists had the ability to reduce expansion and clonogenicity of WT organoids, with the most potent effect observed in combination. Analogously, co-culture of WT organoids with Apc^{-/-} organoids deficient in either Notum, Wif1 or Dkk2 (generated using CRISPR/Cas9) did not rescue WT organoid expansion (FIGS. 7g, h). In addition, CM derived from Wnt-antagonist depleted Apc^{-/-} organoids did not alleviate the reduction in Wnt signalling in our TOP-GFP reporter cell line (FIG. 7i).

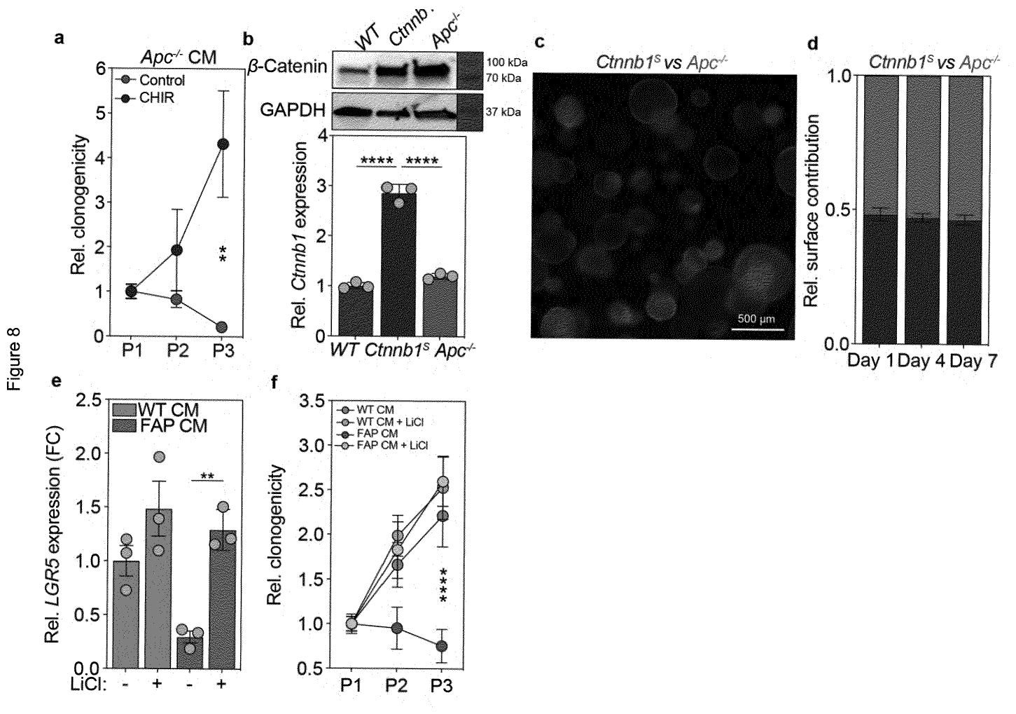

However, titration of the CM from CRISPR/Cas9 KO Apc^{-/-} organoids lacking the three individual factors, established that the cultures deficient in Notum most rapidly lost the ability to reduce clonogenicity in WT organoids (FIG. 7j). Together these data indicate that none of the individual, upregulated Wnt antagonists is solely responsible for the observed inhibitory effects that Apc-deficient cells exert on their neighbours, but that Notum might be most critical in this context. The relevance of NOTUM was also confirmed in human organoids (FIG. 7k, l). Given the central importance of the Wnt pathway in regulation of gut homeostasis, redundancy in molecules controlling the negative feedback is expected. This is in agreement with the accompanying manuscript by Flanagan et al. showing a marked increase in expression of other Wnt antagonists, including Wif1 and Dkk3, after loss of Notum in Apc^{-/-} organoids and adenomas. It was reasoned that rendering WT cells insensitive to the Wnt antagonists by downstream Wnt activation, could provide an effective strategy to reduce the supercompetitor features of Apc-mutant cells. Indeed, organoids which were treated with GSK3β inhibitors LiCl or CHIR, or that expressed a constitutive active variant of β-catenin, were resistant to the Apc^{-/-} CM induced reduction in proliferation and clonogenicity (FIG. 5i, FIGS. 8a-d). Moreover, LiCl administration also rescued loss of sternness and clonogenicity in human colon organoids incubated with FAP CM (FIGS. 8. e-f). This further supports the suggestion that boosting Wnt activation in wild type cells might be a promising approach to limit the competitive benefit of APC-mutant clones in humans.

### Example 4—Downstream Wnt Activation Abrogates Competitive Benefit of Apc-Mutant Cells In Vivo

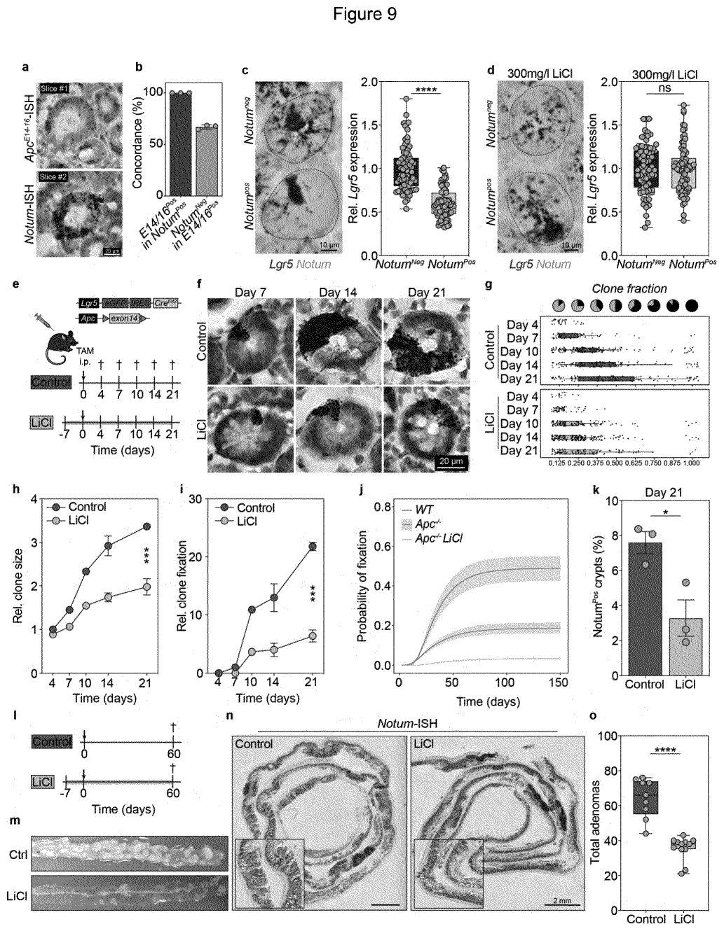

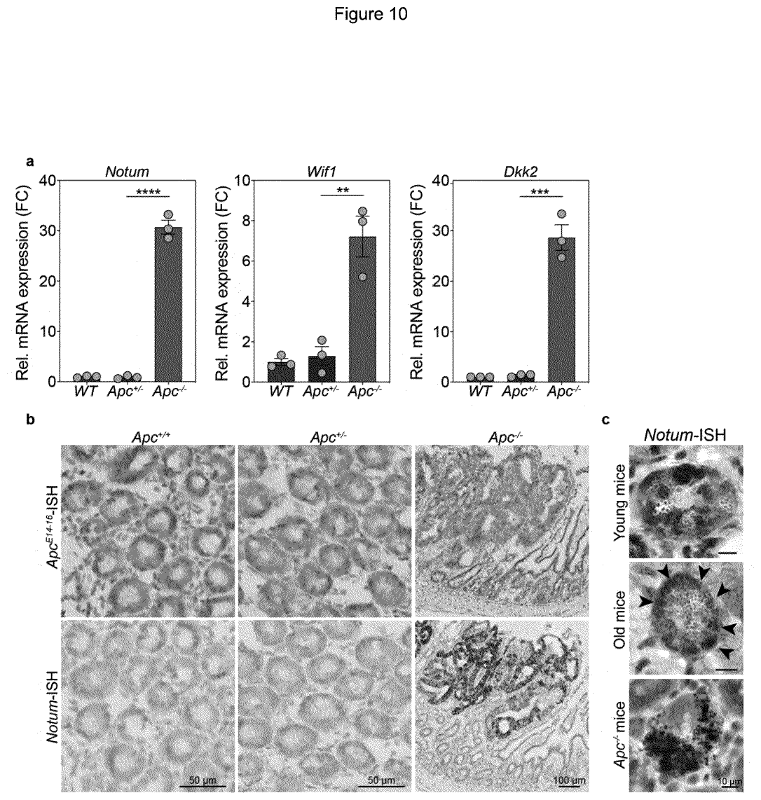

Translation of these in vitro findings to an in vivo model was facilitated by the highly specific upregulation of Notum in Apc-deficient cells (FIGS. 9a, b, FIGS. 10a-c). Analysis of sequential crypt bottom slices using Notum in situ hybridisation showed exclusive expression of Notum in homozygous recombined Apc (Exon14-Exon16) crypts, providing a direct read-out of bi-allelic (Notum^{pos};E14/16^{pos}) Apc loss (FIGS. 9a, b, FIG. 10b). Importantly, the previously reported expression of Notum in (aged) Paneth cells did not impact our analyses due to markedly lower Notum levels in these cells as compared to Apc^{-/-} clones (FIG. 10c)¹¹.

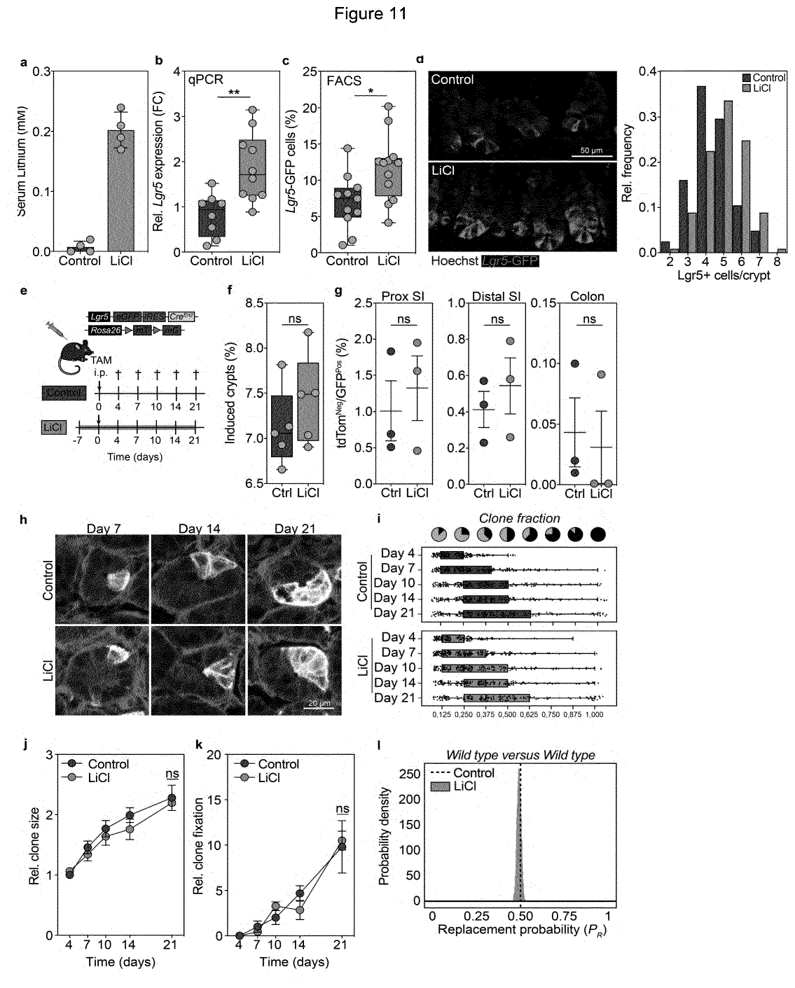

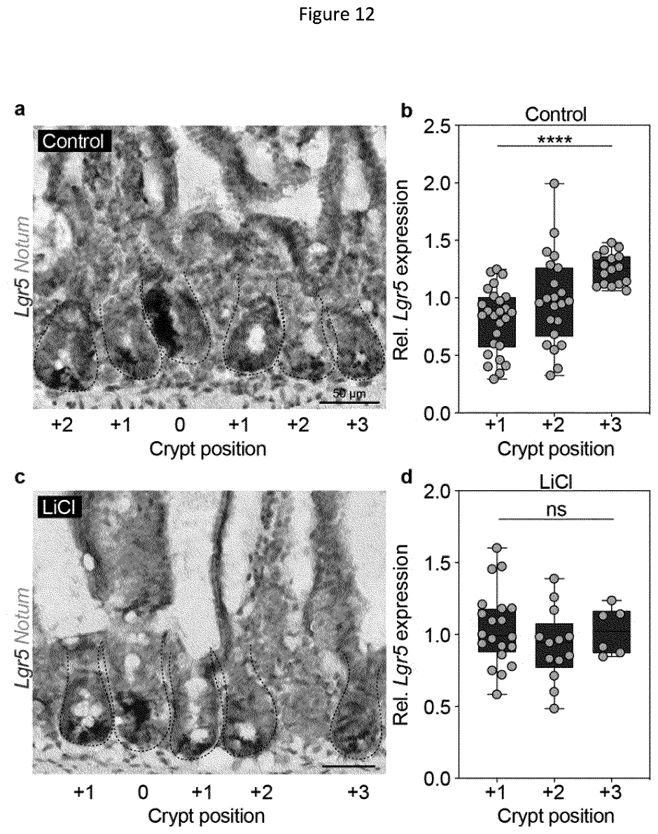

In an accompanying study by Flanagan et al. which is incorporated herein by reference in its entirety it is demonstrated that co-deletion of Notum together with Apc reduces the expansion rate of Apc-mutant clones, suggesting a direct involvement of Wnt antagonists in intestinal transformation. Given the observed functional redundancy in secreted Wnt ligands, it was evaluated if downstream pharmacological activation of the Wnt pathway in WT cells could limit the effects induced in the environment of Apc^{-/-} clones as well as their expansion within the crypt. To this end a model system previously developed to quantify the effect of oncogenic mutations on ISC dynamics in vivo³ was employed. First, it was confirmed that oral LiCl treatment in Lgr5-Cre^{Ert2};Rosa26^{mTmG} mice resulted in well-tolerated serum concentrations and effective Wnt activation in intestinal epithelial cells (FIGS. 11a-d). Moreover, neutral ISC competition in WT mice in the presence or absence of LiCl treatment were studied and it was observed that LiCl had no influence on fundamental ISC dynamics (FIGS. 11e-l). This indicates that LiCl exposed crypts continue to demonstrate neutral drift dynamics. Next, Lgr5-Cre^{Ert2};Ape^{fl/fl} mice were employed and it was detected that while Notum^{pos}/Apc^{-/-} clones reduced Lgr5 expression within the same crypt and in directly neighbouring crypts, LiCl treatment of mice prevented this (FIGS. 9c, d, and FIGS. 12a-d). This both directly confirms the ability of Apc-deficient cells to induce differentiation in vivo, as well as the ability of LiCl to prevent this.

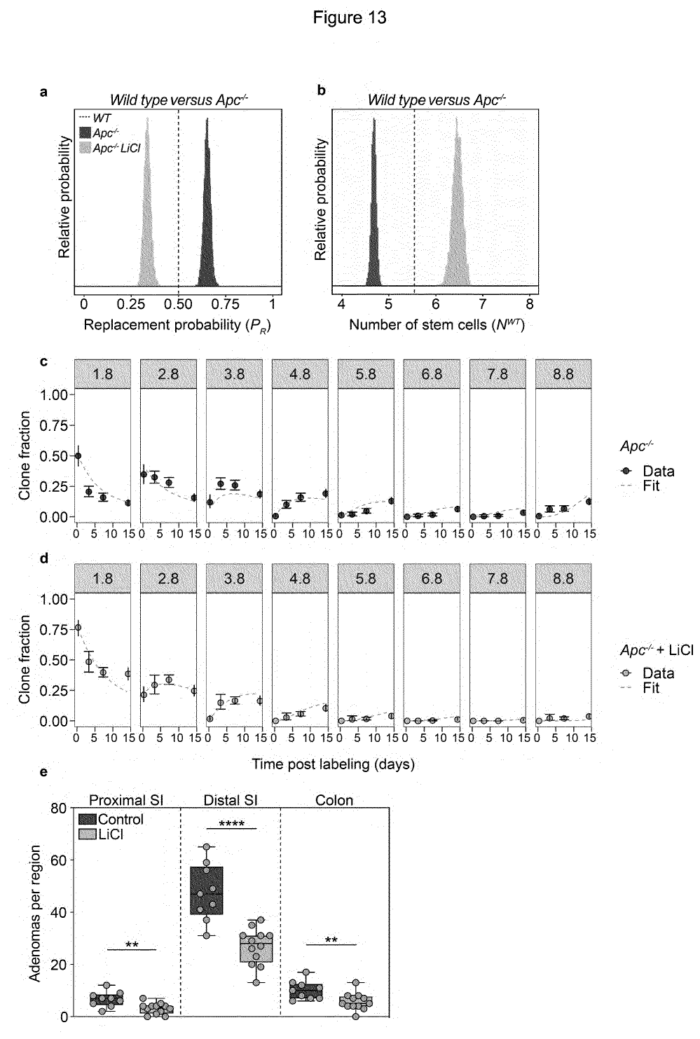

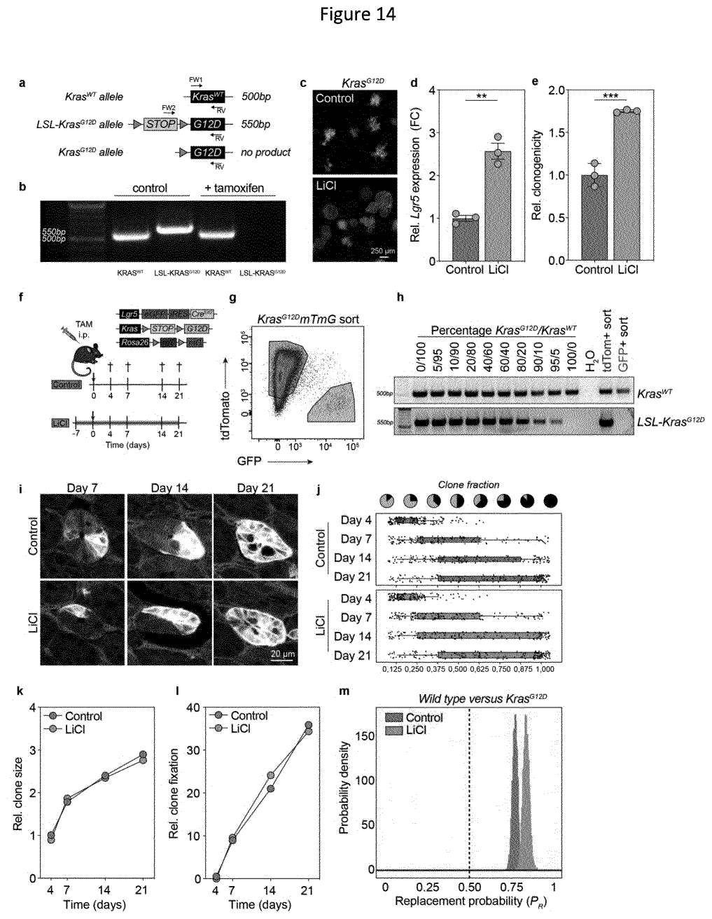

To study the impact of LiCl on the clonal dynamics of Apc-mutant clones, Lgr5-Cre^{Ert2};Apc^{fl/fl} mice were used and Notum^{pos}/Apc^{-/-} clone size distributions within crypt bottoms at predefined days following tamoxifen injection, in the absence or presence of LiCl (FIGS. 9e-g) were evaluated. Treatment with LiCl significantly reduced the rate of Notum^{pos}/Apc^{-/-} clone expansion and fixation compared to the non-treated mice (FIGS. 9h, i). In addition, LiCl reduced the probability of replacement (P_{R}) of WT ISCs by Apc^{-/-} ISCs from 0.65 (95% Cl: 0.62-0.68) to 0.34 (95% Cl: 0.31-0.37) (FIG. 13a). This reduction is even below the initially expected return to neutral competition, corresponding to a P_{R} of 0.5. Further analyses indicated that this is caused by the fact that while Notum^{pos}/Apc^{-/-} clones reduced the number of WT stem cells (N^{WT}) in crypts to an average of 4.7 (95% Cl: 4.5-4.8), as compared to 5.6 (95% Cl: 5.2-5.9) in fully WT crypts, in LiCl treated mice the number of functional ISCs is increased to 6.5 (95% Cl: 6.2-6.8) (FIG. 13b-d). Together, this results in a markedly reduced probability of clonal fixation in a crypt (P_{fix}) (FIG. 9j). This analysis was directly corroborated by a reduced number of Notum^{pos} clones in LiCl treated mice (FIG. 9k). To evaluate if the observed effect of LiCl on mutant ISC dynamics is specific for Apc-deficient clones, and in light of the well-described competitive advantage of Kras^{G12D}_mutant cells^{3,12}, Kras^{G12D}-mutant clones were analysed in vitro and in vivo in the presence or absence of LiCl (FIG. 14). It was found that Kras^{G12D} clone dynamics remained unaffected by treatment with LiCl (FIGS. 14f-m), indicating that the reduction in competitive advantage of Apc-deficient clones is indeed related to antagonizing the specific effect of Apc-mutant clones.

Finally, it was evaluated if the reduction in clonal fixation rate of Apc^{-/-} clones also resulted in a reduction of adenoma formation. To this end, Lgr5-Cre^{Ert2};Apc^{fl/fl} mice were pre-treated with LiCl, induced low-level Apc-inactivation and maintained mice on LiCl treatment.

Sixty days after induction the mice were sacrificed and the number of adenomas was evaluated (FIGS. 9l-n). This experiment revealed markedly reduced adenoma formation in all segments of the intestine (FIG. 90, FIG. 13e), and confirms the potency of rendering WT cells insensitive to the supercompetition effect of Apc-mutant clones in preventing intestinal tumour formation.

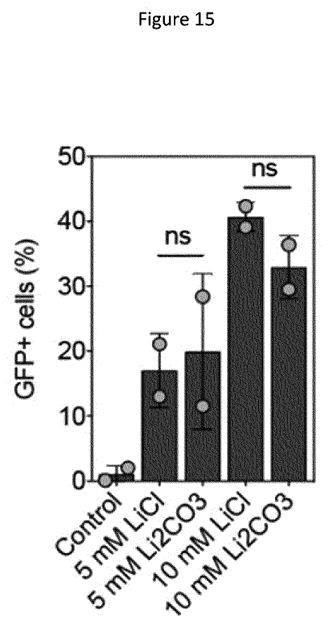

Given the fact that lithium is mostly administered as Li₂CO₃ in the clinic, the inventors assessed whether Li₂CO₃ activates Wnt signalling to a similar degree as LiCl. The effects of both Li₂CO₃ and LiCl were tested using a Wnt reporter assay and revealed that comparable concentrations of Li₂CO₃ and LiCl activated the same amount of Wnt signalling (FIG. 15).

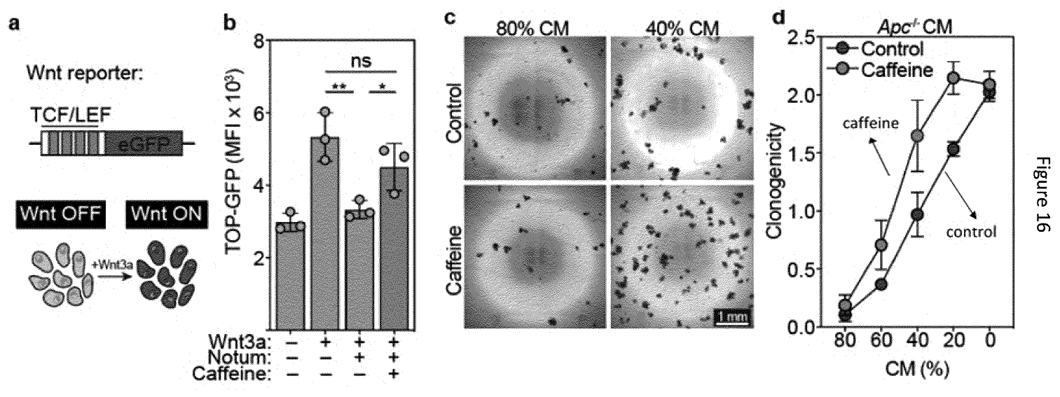

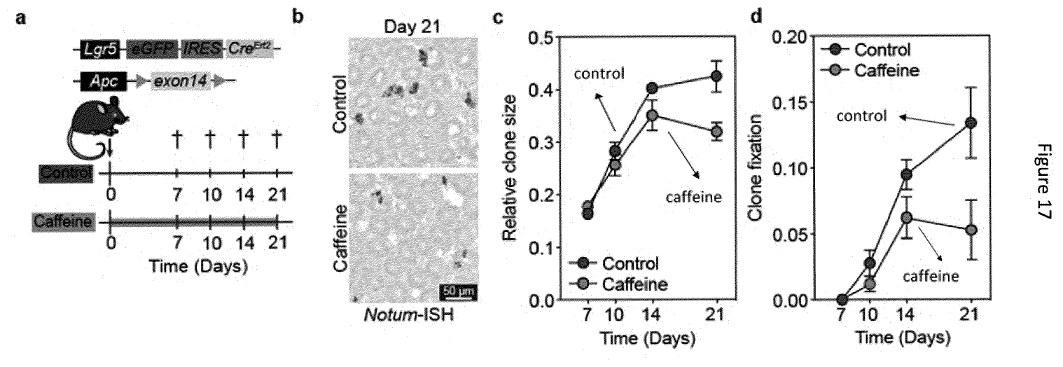

Moreover, a recent study revealed how caffeine is a potent Notum inhibitor, by binding its catalytic pocket and thereby preventing its function. To validate these findings, the inventors first assessed the Notum-inhibiting effect of caffeine in a Wnt reporter assay (FIG. 16a,b). Furthermore, incubation of normal (wild type) intestinal organoids with Apc-mutant CM in the presence of absence of caffeine revealed a rescue in clonogenic capacity of organoids treated with caffeine. In contrast, organoids incubated with the CM of Apc-mutant, Notum KO organoids showed no effect of the caffeine, highlighting that caffeine specifically rescues systems in which Notum is present (FIG. 16c,d). In addition, the in vivo effects of caffeine on clonal dynamics were assessed in a similar way as described in FIG. 9 (FIG. 17a-d). In short, Apc-loss was initiated in mice that were administered caffeine in the drinking water or served as controls (FIG. 17a). Mice were sacrificed at distinct timepoints, and clonal dynamics of Apc mutant cells were determined using Notum-ISH (FIG. 17b). Administration of caffeine resulted in decreased clone size (FIG. 17c) and clone fixation (FIG. 17d). Together these data suggest a promising role for Notum inhibitor caffeine in desensitizing normal cells to Wnt inhibition.

### DISCUSSION

In this study, the inventors have shown that Apc-mutant cells display supercompetitor properties as they actively drive elimination of WT ISCs from the crypt. To date, the best studied examples of supercompetitors are Minute-mutant and Myc-overexpressing cells in Drosophila, which both induce apoptosis in wild type cells¹³⁻¹⁶. In addition, APC-mutant clones in the Drosophila midgut were demonstrated to actively induce apoptosis in the surrounding tissue¹⁷. Here, it was detected that Apc-mutant cells induce differentiation of wild type ISCs through secretion of multiple Wnt antagonists. In agreement, supercompetition by means of differentiation has been described for Drosophila ovarian germline stem cells¹⁸, shown to be important for maintaining tissue integrity during murine skin homeostasis¹⁹, and is proposed to be the main mechanism of competition in adult tissues²⁰. Wnt antagonist Notum has also been determined to be the responsible driver of cell competition in the Drosophila wing imaginal disc²¹. Moreover, secretion of NOTUM by Paneth cells has recently been implicated in reducing stem cell function in the aging intestine, and pharmacological inhibition of NOTUM was shown to rejuvenate the intestine¹¹. Previously, many different Wnt antagonists have been reported to be upregulated in cells following genetic events leading to Wnt activation²²⁻²⁶. In the present work, in conjunction with the study by Flanagan et al., their previously unrecognized contribution to intestinal tumour formation is revealed. Of note, key aspects of the supercompetitor phenotype in a human context are confined. In APC-deficient human cells expression of an even larger set of partially redundant Wnt antagonists including NOTUM, DKK1, SFRP5 and WIF1 was detected (FIG. 5h). This finding is in support of the approach to aim for pharmacological Wnt activation downstream of the ligand-receptor level, e.g. using LiCl.

Materials and Methods

Animal Experiments

Lgr5-EGFP-IRES-Cre^{ERT2} Villin-Cre^{ERT2}, Rosa26mTmG, Apc^{fl/fl} and Kras^{G12D} mice have been described earlier³⁰⁻³⁴. All in vivo experiments were approved by the animal experimentation committee at the Amsterdam UMC—location Academic Medical Center in Amsterdam under nationally registered licence number AVD1180020172125 and performed according to national guidelines. Mice were housed in a 12 hour light/12 hour dark cycle, with temperatures between 20-24° C. and 40-70% humidity. For short term assays, both male and female mice were used. For long term assays, only females were used to prevent the risk of preliminary dropout due to fighting male mice. All mice were between 6-12 weeks old at the start of the experiments. For all mouse experiments, sufficient sample sizes were determined based on previous studies with a similar study design^{3,35}. Experimental animals were randomly assigned to the control or lithium treated groups, clone sizes and adenoma counts were scored blindly. In vivo low-dose recombination was induced by intraperitoneal injection (i.p.) of 0.3 mg (for Rosa26^{mTmG} and Kras^{G12D} mice) or 2 mg (for Ape^{-/-} mice) tamoxifen (Sigma) dissolved in sunflower oil. Mice were either assigned to a control group or treated with lithium chloride (LiCl, Sigma) dissolved at a final concentration of 300 mg/liter in tap water, or with caffeine (Sigma) at a final concentration of 400 mg/liter in tap water. Treatment with LiCl was initiated 7 days before recombination by i.p. injection of tamoxifen and was administered until the day they were sacrificed. For short term experiments to study stem cell dynamics, mice were sacrificed at day 4, 7, 10, 14, 21 days after intra peritoneal (i.p.) injection and intestines were removed and further processed for analyses. For long term adenoma formation experiments, mice were injected with 2 mg tamoxifen and sacrificed 60 days after i.p. injection. Mouse discomfort during tumour formation assays was closely monitored, and endpoints were determined as <15% weight loss within 2 days or a mouse grimace scale (MGS) score<3. These endpoints were not exceeded during this study. After 60 days, intestines were removed and polyps were counted macroscopically.

Tissue Processing & Clone Size Quantification

After the mice were sacrificed, intestines were removed fully and washed thoroughly with ice-cold PBS. The intestines were cut into pieces of 5 mm, opened longitudinally and fixed overnight in 4% paraformaldehyde (PFA) solution. To preserve tissue integrity the intestines were kept in 30% sucrose solution for another night before freezing. Crypt bottoms were sliced with a thickness of 10 m at a Cryostar™ NX70 cryostat and placed on glass slips. Fluorescent lineage tracing labels were visualized with a SP8X Confocal (Leica), slides were counterstained with Hoechst-33342. RNA-ISH stained coupes were counterstained with hematoxylin and scanned using the IntelliSite Ultra Fast 1.6 slide scanner (Philips). For all crypt analyses, clone sizes were quantified as proportions of the crypt circumference (in eights, 1:8-8:8).

Organoid Culture

Murine intestinal crypts were isolated from Lgr5-EGFP-IRES-Cre^{ERT2}, Lgr5-EGFP-IRES-Cre^{ERT2};Ape/or Villin-Cre^{ERT2};Apc^{fl/fl} mice as described by Sato et al.³⁵. In short, intestines were removed from the mouse and washed thoroughly with ice-cold PBS. Next, the intestine was opened longitudinally and the villi were gently scraped off by a glass cover slide. The intestine was cut into pieces of 5×5 mm and incubated in 2 mM EDTA solution for 30 min. at 4° C. After removal of the EDTA, the crypts were resuspended in ice-cold 1% FCS in PBS by vigorously shaking the tube and passing the supernatant through a 70 m strainer. Isolated crypts were resuspended in Matrigel® (Corning) and seeded in pre-heated 24-well plates, supplemented with basal organoid medium consisting of advanced DMEM/F12 medium (Gibco) containing 100× N2 and 50× B27 supplements, 100× Glutamax, 5 mM HEPES, 1 mM N-acetyl-L-cysteine (Sigma), and 100× antibiotic/antimycotic (all Gibco). The basal organoid medium was freshly supplemented with the following growth factors: mouse EGF 50 ng/ml (TEBU-BIO), R-spondin (conditioned medium), Noggin (conditioned medium). The first two days after crypt isolation CHIR99021 (Axon Medchem) and ROCK inhibitor (Sigma) was added to the medium. Ctnnb1^{S} organoids expressing a constitutive active variant of β-catenin were generated as described by Adam et al.³⁶. For in vitro recombination of loxP flanked alleles, 1 μM 40H-Tamoxifen (Sigma) was added to the medium. Recombination of the Apc gene was validated by digital droplet PCR (Bio-Rad) using EvaGreen® Supermix (Bio-Rad). Lgr5-EGFP-IRES-Cre^{ERT2} organoids, referred to as wild type (WT) organoids, were stably transduced with a red fluorescent mCherry construct (LeGO-C2, Addgene #27399). The in vitro recombined Lgr5-EGFP-IRES-Cre^{ERT2};Apc^{fl/fl}, referred to as Apc^{-/-}, were stably transduced with a green fluorescent Venus construct (LeGO-V2, Addgene #27340). During competition assays, organoids were plated in equal numbers in 24-well plates and full wells were scanned over time by the EVOS FL Cell Imaging System (Thermo Scientific). Conditioned medium (CM) was taken from 2-3 day old WT or Apc^{-/-} organoid cultures and freshly supplemented with growth factors R-spondin, Noggin and mEGF. During the competition assays the medium (normal and conditioned) was replaced every other day to minimize effects of medium depletion. To assess the effect of medium depletion, CM was 10× concentrated using 10 kDa Amicon® centrifugal filters (Millipore) and added to fresh ENR medium as 1:10. GSK3β inhibition or Notum inhibition in the CM transfer assays was performed by administering 5 mM LiCl (GSK3p inhibitor), 2.5 μM CHIR99021 (GSK3p inhibitor), or 200 μM caffeine (Notum inhibitor) to the medium. Recombinant proteins NOTUM (2 μg/mL, R&D, 9150-NO-050), WIF1 (5 μg/mL, R&D, 135-WF-050) and DKK2 (1 μg/mL, R&D, 2435-DKB-010) were freshly added to the culture medium, medium was refreshed every other day.

Human organoid cultures were derived from normal colonic tissue obtained from resection material and from polyps of patients diagnosed with familial adenomatous polyposis (FAP)³⁷. The collection of normal and adenomatous material from the colon was approved by the Medical Ethical Committee of Academic Medical Center (AMC), under approval numbers 2014/178 (normal tissue) and MEC 09/146 (adenoma tissue). Normal colon organoids were isolated and processed as previously described⁶. FAP organoids were generated by cutting the polyps into small pieces and plating them into Matrigel® (Corning) and were described previously³⁷. Both normal and FAP organoids were cultured in basal organoid medium as described above, freshly supplemented with 10 mM Nicotinamide (Sigma), 10 μg/mL gentamicin (Lonza), 3 μM SB202190 (Sigma), 500 nM A83-01 (Tocris), 10 nM Prostaglandin E2 (Santa Cruz Biotechnology), 10 nM Gastrin (Sigma), 20 ng/mL human EGF (Peptrotech), R-spondin and Noggin. Normal colon organoids were additionally supplemented with Wnt3a (conditioned medium). Medium was refreshed every 2 days.

CRISPR Cloning

To generate CRISPR KO lines for Notum, Wif1 and Dkk2, two different sgRNA's were designed for each gene using Benchling and sequences can be found in Table 1. The sgRNA oligos were cloned into the lentiCRISPR v2 plasmid (Addgene #52961) and transformed using Stabl3 competent bacteria (Invitrogen). Successful cloning of the guides was verified using Sanger sequencing. Lentiviral particles were generated using third generation packaging plasmids pMDLg/pRRE (Addgene #12251), pRSV-Rev (Addgene #12253) and MD2.G (Addgene #12259). Organoids were transduced with plasmids containing viral particles containing two sgRNA's for one gene, to accommodate the disruption of the target gene through large editing events. After puromycin selection, organoids were single cell sorted to generate unique KO clones, that were validated for editing by Sanger sequencing and TIDE analysis³⁸.

TABLE 1
CRISPR sgRNA sequences

| | | | SEQ |
|---|---|---|---|
| sgRNA | Exon | Sequence | ID NO: |
| Notum sgRNA1 | 1 | TCACCTCCTGCTGAACACGT | 1 |
| Notum sgRNA 2 | 2 | TACTATTTGAAGGAGTCCAA | 2 |
| Wif1 sgRNA 1 | 1 | AGAGCCGGAGCGCGAAAGCA | 3 |
| Wif1 sgRNA 2 | 2 | ACATTCTGATTGTCTCGGAG | 4 |
| Dkk2 sgRNA 1 | 1 | ATCCTTGACCCGCATCAGCG | 5 |
| Dkk2 sgRNA 2 | 1 | CTCACAGCTAGGCAGCTCGC | 6 |

Cell Culture

Mouse embryonic fibroblasts (MEFs, ATCC) and HEK293T (ATCC) cells were both cultured in Dulbecco's Modified Eagle's Medium (DMEM) supplemented with 10% fetal calf serum (FCS), 1% Glutamine, and antibiotic penicillin and streptomycin. Cells were maintained at 37° C. in humidified air containing 5% CO₂. All cell lines were routinely checked for mycoplasm contamination, no cell line authentication was performed.

Generation of Overexpression Constructs

To generate overexpression lines for Notum, Wif1 and Dkk2 RNA was isolated from Apc-mutant intestinal organoids. cDNA was generated using SuperScript Ill RT (Sigma) and ORFs for Notum, Wif1 and Dkk2 were PCR amplified using primers containing Ecorl and Notl restriction digestion sites. Primer sequences can be found in Table 2. Amplified ORFs were cloned into lentiviral plasmid LegO-V2 (Addgene #27340). Lentiviral particles were generated as described above. The viral particles were transduced into HEK293T cells and Venus-positive cells were selected by FACS sorting. Expression levels of Notum, Wif1 and Dkk2 were assessed using RT-qPCR and protein levels were validated using ELISA for NOTUM (LS-F17999) and WIF1 (LS-F39936-1, LS Biosciences).

TABLE 2
mORF sequences for OE constructs. Restriction digestion sites are underlined

| | | | SEQ |
|---|---|---|---|
| Name | | Sequence | ID NO: |
| Notum ORF | FW | GACGACGAATTCGCCACCATGGGAGGAGAGGTGCGCGTG | 7 |
| EcoRI | | CTGCTACTG | |
| Notum ORF | RV | GACGACGCGGCCGCCTAGTTCCCATTACTCAGCATCCCTA | 8 |
| NotI RV | | GC | |
| Wif1 ORF NoI | FW | GACGACGCGGCCGCGCCACCATGGCTCGGAGAAGAGCC | 9 |
| | | TTCCCTGCTTTCGC | |
| Wif1 ORF NotI | RV | GACGACGCGGCCGCTCACCAGATGTAATTGGATTCAGGT | 10 |
| RV | | GGATCC | |
| Dkk2 ORF | FW | GACGACGAATTCGCCACCATGGCCGCGCTGATGCGGGTC | 11 |
| EcoRI | | AAGGATTCA | |
| Dkk2 ORF NotI | RV | GACGACGCGGCCGCTCAGATCTTCTGGCATACATGGAGT | 12 |
| | | CTG | |

TOP-GFP Assay

Mouse embryonic fibroblasts (MEFs) were cultured in Dulbecco's Modified Eagle's Medium (DMEM) supplemented with 10% fetal calf serum (FCS), 1% Glutamine, and antibiotic penicillin and streptomycin. MEFs were stably transduced with Wnt reporter TOP-GFP (Addgene plasmid #35491). For TOP-GFP assays, cells were stimulated with Wnt3A conditioned medium for 24 hours after which GFP positivity was measured by flow cytometry. For downstream Wnt activation, either 5 or 10 mM LiCl, 5 or 10 mM Li₂CO₃, 2.5 μM CHI R99021 or 200 μM caffeine was supplemented to the medium.

RNA Isolation & ciPCR

RNA was extracted using the Bioke Nucleospin RNA isolation kit (cat no. 740955). Complementary DNA syntheses was generated using SuperScript Ill RT (Sigma). Sybr Green (Roche) RT-qPCR reactions were performed with the Roche LightCycler 480 system under standard conditions. The ΔΔCt method was used to calculate gene expression. All AACt values were normalized to housekeeping genes RpI37 and Hprt. Primer sequences can be found in Table 3.

TABLE 3
qPCR primer sequences

| Gene | Species | FW/RV | Sequence | SEQ ID NO: |
|---|---|---|---|---|
| Alpi | Mouse | FW | GGCTACACACTTAGGGGGACCTCCA | 13 |
| | | RV | AGCTTCGGTGACATTGGGCCGGTT | 14 |
| Ascl2 | Mouse | FW | CTACTCGTCGGAGGAAAG | 15 |
| | | RV | ACTAGACAGCATGGGTAAG | 16 |
| Axin2 | Mouse | FW | CCATGACGGACAGTAGCGTA | 17 |
| | | RV | CTGCGATGCATCTCTCTCTG | 18 |
| ChgA | Mouse | FW | AAGAAGAGGAGGAGGAAGAGG | 19 |
| | | RV | TCCATCCACTGCCTGAGAG | 20 |
| Dkk2 | Mouse | FW | TCAGTCAGCCAACCGATCTG | 21 |
| | | RV | TCTCTGTGGCATCGTTTCTTTT | 22 |
| Hprt | Mouse | FW | TGTAATGATCAGTCAACGGGGG | 23 |
| | | RV | AGAGGTCCTTTTCACCAGCAA | 24 |
| Krt20 | Mouse | FW | GCTGCAAAATCAGGTTAAGGA | 25 |
| | | RV | TCCCCTCTCAGTCTCAAACTTC | 26 |
| Lgr5 | Mouse | FW | TTCGTAGGCAACCCTTCTCT | 27 |
| | | RV | TCCTGTCAAGTGAGGAAATTCA | 28 |
| Lyz1 | Mouse | FW | GCCAAGGTCTACAATCGTTGTGAGTTG | 29 |
| | | RV | CAGTCAGCCAGCTTGACACCACG | 30 |
| Muc2 | Mouse | FW | CTGACCAAGAGCGAACACAA | 31 |
| | | RV | CATGACTGGAAGCAACTGGA | 32 |
| Notum | Mouse | FW | CTGCGTGGTACACTCAAGGA | 33 |
| | | RV | CCGTCCAATAGCTCCGTATG | 34 |
| Rpl37 | Mouse | FW | CCAAGGCCTACCACCTTCAG | 35 |
| | | RV | CAGTCCCGGTAGTGTTTCGT | 36 |
| Wif1 | Mouse | FW | CAAAGAATGCCAGCCATTCC | 37 |
| | | RV | CAGCAAAGGGACATTGACAG | 38 |
| GAPDH | Human | FW | AATCCCATCACCATCTTCCA | 39 |
| | | RV | TGGACTCCACGACGTACTCA | 40 |
| GUSB | Human | FW | TGGTTGGAGAGCTCATTTGGA | 41 |
| | | RV | GCACTCTCGTCGGTGACTGTT | 42 |
| KRT20 | Human | FW | TCCTCAAAAAGGAGCATCAGGAG | 43 |
| | | RV | TGATGACGCCAAGGTTCAGG | 44 |
| LGR5 | Human | FW | ACCAGACTATGCCTTTGGAAAC | 45 |
| | | RV | TTCCCAGGGAGTGGATTCTAT | 46 |
| MUC2 | Human | FW | ACCCGCACTATGTCACCTTC | 47 |
| | | RV | GCATCATATGCACGGTCTTG | 48 |
| NOTUM | Human | FW | ACTCGCACAGGCACAGGGA | 49 |
| | | RV | GCCCCGCTCCAAACATCACT | 50 |

Western Blotting

Organoids were harvested in Cell Recovery solution and incubated on ice for 30 min to remove Matrigel remnants. Following 2 PBS washes, protein lysates were made using 10× cell lysis buffer (Cell Signaling Technologies) according to manufactureres' protocol. Protein concentrations were determined using Pierce™ Protein Assay Kit (Thermo Scientific), and 30 μg protein was loaded in 4-15% Mini-PROTEAN® TGX precast protein gels (Bio-Rad), separated by electrophoresis and transferred to PDVF membranes using the Trans-Blot Turbo System (Bio-Rad). Next, membranes were blocked in 5% Skim Milk Powder (Sigma) before they were incubated with primary antibodies in 5% BSA/TBST overnight at 4° C. on a roller bank. The next day, the membranes were incubated with HRP-conjugated secondary antibodies for 1 h at room temperature in 5% BSA/TBST. Protein levels were detected using Pierce™ ECL Western Blotting Substrate (Thermo Scientific) and revealed using ImageQuant LAS 4000 (GE Healthcare Life Sciences). Primary antibodies used are anti-β-Catenin (9562, Cell Signaling Technologies) and anti-GAPDH (MAB374, Millipore). Secondary antibodies are anti-rabbit-HRP (7074, Cell Signaling technologies) and anti-mouse-HRP (1070-05, Southern Biotech).

Flow Cytometry

All FACS analysis experiments were performed on the BD LSRFortessa™ (BD Biosciences). FACS sorting was performed on the BD FACSAria™ III Cell Sorter (BD Biosciences). Lgr5-GFP^{high} populations were gated on Dapi^{neg} population. Absolute cell numbers were determined using BD Trucount tubes (BD Biosciences). Data acquisition was performed using FACSDiva software V8 (BD Biosciences), data analysis was performed using FlowJo software (Flowjo, LLC).

Immunofluorescence

Murine stainings were performed on fixed frozen and paraffinized tissues. Human stainings were performed on paraffined biopsies derived from FAP patients, all biopsies were scored by a pathologist for adenomatous lesions. Prior to staining, paraffin coupes were deparaffinized and treated with antigen retrieval in citrate solution (pH 6.0). Next, samples were blocked using ultra-V blocking solution (Immunologic). Primary antibodies were administered in antibody diluent (ScyTek) and incubated overnight at 4° C. Slides were washed thoroughly and incubated in secondary antibody for 1 h at room temperature. Hoechst-33342 (Thermo Scientific) was used as nuclear counterstain and was incubated at 10 g/ml for 5 min at room temperature before slides were covered with Prolong™ Gold antifade reagent (Invitrogen) and sealed with coverslips (VWR). All stainings were analysed using the SPX8 Confocal (Leica) and stored at 4° C. The following antibodies were used: anti-mouse MUC2 (sc-15334, Santa Cruz, 1:100), anti-human E-Cadherin (AF748, R&D Systems, 1:200), anti-rabbit Alexa Fluor 488 (A11034, Invitrogen, 1:500), and anti-goat Alexa Fluor 488 (51475A, Invitrogen, 1:500).

RNA In Situ Hybridisation

RNA in situ hybridisation (RNAscope) and Basescope was performed on fixed frozen intestinal tissue (mouse) and paraffine embedded tissue (mouse and human) according to manufacturer's protocol (ACD RNAscope® 2.5 HD—Brown and Red, and ACD BaseScope™ v2—Red). RNAscope was used for detection of mouse Notum (#428981), Wif1 (#412361), Dkk2 (#404841) or positive control Ppib (#313911) and human NOTUM (#430311) and positive control PPIB (#313901). BaseScope probe ApcE14-E16 (#703011) was used to detect recombined Apc alleles. RNAscope duplex was performed using the RNAscope Duplex Reagent Kit (#322430, ACD) with additional Lgr5 probe (#312171-C2). After RNAscope procedures tissues were counterstained for Hematoxylin or Hoechst-33342. RNAscope was quantified using QuPath software v0.2.2³⁹.

RNA-Seq

Organoid RNA sequencing libraries were prepared using the KAPA RNA Hyperprep with RiboErase (Roche) following manufacturer's protocol. Total RNA isolation was performed by trizol-chloroform extraction in combination with the RNeasy MinElute Cleanup Kit (Qiagen, Hilden, Germany). RNA integrity was assessed with the Agilent 2100 Bioanalyzer (Agilent Technologies, CA, USA). Libraries were barcoded, quantified using NEBNext® Library Quant Kit for Illumina (New England Biolabs (NEB), MA, USA), pooled equimolarly and multiplex sequenced (single-end 50 bp reads) on the Illumina Hiseq4000 platform.

RNA-Seq Data Analysis

Sequence read quality was assessed by means of the FastQC method (v0.11.5; http://www.bioinformatics.babraham.ac.uk/proiects/fastqc/). Trimmomatic version 0.36 was used to trim Illumina adapters and poor-quality bases (trimmomatic parameters: leading=3, trailing=3, sliding window=4:15, minimum length=40)⁴⁰. The remaining high-quality reads were used to align against the Genome Reference Consortium mouse genome build 38 (GRCm38)⁴¹. Mapping was performed by HISAT2 version 2.1.0 with parameters as default⁴². Count data were generated by means of the HTSeq method, and analysed using the DESeq2 method in the R statistical computing environment (R Core Team 2014. R: A language and environment for statistical computing. R Foundation for Statistical Computing, Vienna, Austria)^{43,44}. Statistically significant differences were defined by Benjamini & Hochberg adjusted probabilities<0.05.

Data Visualization

Heatmaps and Volcano plots from publicly available datasets GSE145308⁴⁵, GSE6546⁴⁶ and GSE8671⁴⁷ were analysed using Genomics Analysis and Visualization Platform R2⁴⁸. Signatures scores were based on published gene signatures for Wnt signalling “HALLMARK_WNT_BETA_CATENIN_SIGNALING” (M5895), van der Flier⁴⁹ and intestinal stem cells signatures by Munoz⁵⁰ and Merlos-Suerez⁵¹.

### Stem Cell Drift Modelling

The clone data was modelled using the models and methods developed in Vermeulen et al.³. An R package implementing the model was used and is available at https://qithub.com/MorrisseyLab/CryptDriftR. Briefly, the clonal dynamics generated by the stem cells are modelled as a one-dimensional discrete random walk with absorbing states at O and N, where N is the total number of stem cells^{3,52}. When modelling mutant stem cells, the balance between replacing neighbours or being replaced is inferred directly from the data. The fitting of the stochastic model to the data is done using a Bayesian approach with a multinomial likelihood for the counts of the different clone sizes measured.

In light of experimental observations on stem cell numbers the model was adapted to include the change in WT stem cell numbers as a mode of mutant advantage. The same base drift model was utilised, however to add granularity the change in stem cell numbers to be distributional was considered rather than a single change. This is done by modelling the full distribution as a mixture of drift models with different numbers of stem cells, where the mixing weights are to be inferred.

$$Q ( λ , τ , t ) = {\sum }_{n = 3}^{{N}_{max}} {α}_{n} {P}_{n} ( λ , τ , t )$$

In order to constrain the degrees of freedom of the model an was linked by modelling αₙ as a gaussian with a mean and a variance to be inferred and later normalising so that Σαᵢ=1. The model is implemented in Stan and distributions are produced using HMC⁵³.

Statistics and Reproducibility

All in vitro organoid monocultures were quantified blindly using ImageJ FIJI v2.0⁵⁴. This was impossible for the co-culture experiments due to the fluorescent features of these co-cultures. All in vivo histological data was scored blindly. Visualization and statistical analysis of data was performed using Graphpad Prism, where most data was analysed using two-sided Student's t-test. In case other statistical tests were applied this was noted in the figure legends. All RNAscopes were performed on at least 3 independent biological samples (either 3 different mice or patients).

Data Availability

The sequence libraries that support the findings in this study are publicly available through the National Center for Biotechnology Information (NCBI) Gene Expression Omnibus (GEO) under accession GSE144325: https://www.ncbi.nlm.nih.gov/qeo/query/acc.cgi?acc=GSE144325

Throughout the description and claims of this specification, the words “comprise” and “contain” and variations of them mean “including but not limited to”, and they are not intended to (and do not) exclude other components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

Each document, reference, patent application or patent cited in this text is expressly incorporated herein in their entirety by reference, which means it should be read and considered by the reader as part of this text. That the document, reference, patent application or patent cited in the text is not repeated in this text is merely for reasons of conciseness. Reference to cited material or information contained in the text should not be understood as a concession that the material or information was part of the common general knowledge or was known in any country.

### REFERENCES

3. Vermeulen, L. et al. Defining stem cell dynamics in models of intestinal tumor initiation. Science 342, 995-8 (2013).
4. Fearon, E. F. & Vogelstein, B. for Colorectal Tumorigenesis. Cell 61, 759-767 (1990).
5. Morin, P. J., Vogelstein, B. & Kinzler, K. W. Apoptosis and APC in colorectal tumorigenesis. Proc. Natl. Acad. Sci. (2002). doi:10.1073/pnas.93.15.7950
6. Fevr, T., Robine, S., Louvard, D. & Huelsken, J. Wnt/beta-catenin is essential for intestinal homeostasis and maintenance of intestinal stem cells. Mol. Cell. Biol. (2007). doi:10.1128/MCB.01034-07
7. Boone, P. G. et al. A cancer rainbow mouse for visualizing the functional genomics of oncogenic clonal expansion. Nat. Commun. 10, 5490 (2019).
8. Albuquerque, C. et al. The ‘just-right’ signaling model: APC somatic mutations are selected based on a specific level of activation of the beta-catenin signaling cascade. Hum. Mol. Genet. (2002). doi:10.1093/hmg/11.13.1549
9. Crabtree, M. et al. Refining the relation between ‘first hits’ and ‘second hits’ at the APC locus: The ‘loose fit’ model and evidence for differences in somatic mutation spectra among patients. Oncogene (2003). doi:10.1038/sj.onc.1206471
10. Haber, A. L. et al. A single-cell survey of the small intestinal epithelium. Nature (2017). doi:10.1038/nature24489
11. Pentinmikko, N. et al. Notum produced by Paneth cells attenuates regeneration of aged intestinal epithelium. Nature (2019). doi:10.1038/s41586-019-1383-0
12. Snippert, H. J., Schepers, A. G., Van Es, J. H., Simons, B. D. & Clevers, H. Biased competition between Lgr5 intestinal stem cells driven by oncogenic mutation induces clonal expansion. EMBO Rep. 15, 62-69 (2014).
13. Morata, G. & Ripoll, P. Minutes: Mutants of Drosophila autonomously affecting cell division rate. Dev. Biol. (1975). doi:10.1016/0012-1606(75)90330-9
14. de la Cova, C. et al. Drosophila Myc Regulates Organ Size by Inducing Cell Competition. 117, 107-116 (2004).
15. Moreno, E., Basler, K. & Morata, G. Cells compete for decapentaplegic survival factor to prevent apoptosis in Drosophila wing development. Nature (2002). doi:10.1038/416755a
16. Moreno, E. & Basler, K. dMyc transforms cells into super-competitors. Cell (2004). doi:10.1016/S0092-8674(04)00262-4
17. Suijkerbuijk, S. J. E., Kolahgar, G., Kucinski, I. & Piddini, E. Cell Competition Drives the Growth of Intestinal Adenomas in Drosophila. Curr. Biol. 26, 428-438 (2016).
18. Jin, Z. et al. Differentiation-Defective Stem Cells Outcompete Normal Stem Cells for Niche Occupancy in the Drosophila Ovary. Cell Stem Cell (2008). doi:10.1016/j.stem.2007.10.021
19. Liu, N. et al. Stem cell competition orchestrates skin homeostasis and ageing. Nature (2019). doi:10.1038/s41586-019-1085-7
20. Ellis, S. J. et al. Distinct modes of cell competition shape mammalian tissue morphogenesis. Nature (2019). doi:10.1038/s41586-019-1199-y
21. Vincent, J. P., Kolahgar, G., Gagliardi, M. & Piddini, E. Steep Differences in Wingless Signaling Trigger Myc-Independent Competitive Cell Interactions. Dev. Cell (2011). doi:10.1016/j.devcel.2011.06.021
22. Kakugawa, S. et al. Notum deacylates Wnt proteins to suppress signalling activity.

Nature (2015). doi:10.1038/nature14259
23. Koo, B. K. et al. Tumour suppressor RNF43 is a stem-cell E3 ligase that induces endocytosis of Wnt receptors. Nature (2012). doi:10.1038/nature11308
24. De Robertis, M. et al. Novel insights into Notum and glypicans regulation in colorectal cancer. Oncotarget (2015). doi:10.18632/oncotarget.5652
25. González-Sancho, J. M. et al. The Wnt antagonist DICKKOPF-1 gene is a downstream target of β-catenin/TCF and is downregulated in human colon cancer. Oncogene (2005). doi:10.1038/sj.onc.1208303
26. Niida, A. et al. DKK1, a negative regulator of Wnt signaling, is a target of the β-catenin/TCF pathway. Oncogene (2004). doi:10.1038/sj.onc.1207892
30. Barker, N. et al. Identification of stem cells in small intestine and colon by marker gene Lgr5. Nature 449, 1003-1007 (2007).
31. El Marjou, F. et al. Tissue-specific and inducible Cre-mediated recombination in the gut epithelium. Genesis (2004). doi:10.1002/gene.20042
32. Shibata, H. et al. Rapid colorectal adenoma formation initiated by conditional targeting of the APC gene. Science (80-.). 278, 120-133 (1997).
33. Muzumdar, M. D., Tasic, B., Miyamichi, K., Li, N. & Luo, L. A global double-fluorescent cre reporter mouse. Genesis (2007). doi:10.1002/dvg.20335
34. Johnson, L. et al. Somatic activation of the K-ras oncogene causes early onset lung cancer in mice. Nature (2001). doi:10.1038/35074129
35. Sato, T. et al. Long-term expansion of epithelial organoids from human colon, adenoma, adenocarcinoma, and Barrett's epithelium. Gastroenterology (2011). doi:10.1053/j.gastro.2011.07.050
36. Adam, R. S. et al. Intestinal region-specific Wnt signalling profiles reveal interrelation between cell identity and oncogenic pathway activity in cancer development. Cancer Cell Int. (2020). doi:10.1186/s12935-020-01661-6
37. Fessler, E. et al. TGFβ signaling directs serrated adenomas to the mesenchymal colorectal cancer subtype. EMBO Mol. Med. (2016). doi:10.15252/emmm.201606184
38. Brinkman, E. K., Chen, T., Amendola, M. & Van Steensel, B. Easy quantitative assessment of genome editing by sequence trace decomposition. Nucleic Acids Res. (2014). doi:10.1093/nar/gku936
39. Bankhead, P. et al. QuPath: Open source software for digital pathology image analysis. Sci. Rep. (2017). doi:10.1038/s41598-017-17204-5
40. Bolger, A. M., Lohse, M. & Usadel, B. Trimmomatic: A flexible trimmer for Illumina sequence data. Bioinformatics (2014). doi:10.1093/bioinformatics/btu170
41. Harrow, J. et al. GENCODE: producing a reference annotation for ENCODE. Genome Biol. (2006).
42. Kim, D., Langmead, B. & Salzberg, S. L. HISAT: A fast spliced aligner with low memory requirements. Nat. Methods (2015). doi:10.1038/nmeth.3317
43. Anders, S., Pyl, P. T. & Huber, W. HTSeq-A Python framework to work with high-throughput sequencing data. Bioinformatics (2015). doi:10.1093/bioinformatics/btu638
44. Love, M. I., Huber, W. & Anders, S. Moderated estimation of fold change and dispersion for RNA-seq data with DESeq2. Genome Biol. (2014). doi:10.1186/s13059-014-0550-8
45. Ringel, T. et al. Genome-Scale CRISPR Screening in Human Intestinal Organoids Identifies Drivers of TGF-β Resistance. Cell Stem Cell (2020). doi:10.1016/j.stem.2020.02.007
46. Reed, K. R. et al. Hunk/Mak-v is a negative regulator of intestinal cell proliferation. BMC Cancer (2015). doi:10.1186/s12885-015-1087-2
47. Sabates-Bellver, J. et al. Transcriptome profile of human colorectal adenomas. Mol. Cancer Res. (2007). doi:10.1158/1541-7786.MCR-07-0267
48. R2 database. Genomic Analysis and Visualization Platform. The Department of Human Genetics in the Amsterdam Medical Centre (AMC) (2019).
49. Van der Flier, L. G. et al. The Intestinal Wnt/TCF Signature. Gastroenterology (2007). doi:10.1053/j.gastro.2006.08.039
50. Muñoz, J. et al. The Lgr5 intestinal stem cell signature: Robust expression of proposed quiescent ‘+4’ cell markers. EMBO J. (2012). doi:10.1038/emboj.2012.166
51. Merlos-Suerez, A. et al. The intestinal stem cell signature identifies colorectal cancer stem cells and predicts disease relapse. Cell Stem Cell 8, 511-524 (2011).
52. Lopez-Garcia, C., Klein, A. M., Simons, B. D. & Winton, D. J. Intestinal stem cell replacement follows a pattern of neutral drift. Science 330, 822-825 (2010).
53. Carpenter, B. et al. Stan: A probabilistic programming language. J. Stat. Softw. (2017). doi:10.18637/jss.v076.i01
54. Schindelin, J. et al. Fiji: An open-source platform for biological-image analysis. Nature Methods 9, 676-682 (2012).

1. A Wnt agonist for use in preventing gastrointestinal tract cancer in a subject, wherein the subject has a genetic predisposition to gastrointestinal tract cancer.

2. The Wnt agonist for the use of claim 1, wherein the cancer is a stomach, intestinal, colon or rectal cancer.

3. The Wnt agonist for the use of any preceding claim, wherein Wnt agonist inhibits the expansion of constitutively Wnt antagonist expressing cells.

4. A Wnt agonist for use in preventing or inhibiting the formation of intestinal pre-cancerous lesions or intestinal adenoma, wherein the Wnt agonist prevents or inhibits expansion of constitutively Wnt antagonist expressing cells in a subject.

5. The Wnt agonist for use according to any preceding claim, wherein the subject has been diagnosed with Familial Adenomatous Polyposis (FAP) or attenuated FAP.

6. The Wnt agonist for use according to any preceding claim wherein the subject has an APC mutation or a β-catenin mutation, optionally wherein the APC mutation is a germline APC gene mutation.

7. The Wnt agonist for use according to any preceding claim, wherein the Wnt agonist is one or more of: a surrogate wnt; chir; R-Spondin analogue; wnt3a; Wnt 5; Wnt-6a; Norrin; CHIR99021; LiCl; BIO-Acetoxime; CHIR 98014; GSK-3 inhibitor IV; SB216763; SB415286; 5-ethyl-7,8-dimethoxy-1H-pyrrolo [3,4-c]-Isoquinoline-1,3-(2H)-dione “3F8”; 9-bromo-7,12-dihydro-indolo [3,2-d][1 Benzazepine-6 (5H)-one “kenpaullone”; 9-bromo-7,12-dihydro-pyrido [3′, 2′: 2,3 Azepino [4,5-b]indol-6 (5H)-one “1-Azakenpaullone”; N-(3-chloro-4-methylphenyl)-5-(4-nitrophenyl)-1,3,4-oxaxe Diazole-2-amine “TC-G24”; 2-methyl-5-[3-[4-(methylsulfinyl) phenyl]-5-benzofuranyl]-1,3,4-Oxadiazole “TCS 2002”; N-[(4-methoxyphenyl) methyl]-N′-(5-nitro-2-thiazolyl) urea “AR-A014418”; 3-[5-[4-(2-hydroxy-2-methyl-1-oxopropyl)-1-piperazinyl]-2-(trifluoromethyl) phenyl]-4-(1H-indole-3-YI)-1H-pyrrole-2,5-dione “TCS 21311”; 3-[[6-(3-aminophenyl)-7H-pyrrolo [2,3-d]pyrimidin-4-yl]oxy]-phenol “TWS119”; 4-(2-Amino-4-oxo-2-imidazolin-5-ylidene)-2-bromo-4,5,6,7-tetrahydropyrrolo [2,3-c]azepine-8-one “10Z-hymenialdicine”; 1997), 2-[(3-iodophenyl) methylsulfanyl]-5-pyridin-4-yl-1,3,4-oxadiazole “GSK-3 beta inhibitor II”; G 4-benzyl-2-methyl-1,2,4-thiadiazolidine-3,5-dione; FRATtide peptide; 3-amino-1H-pyrazolo [3,4-b]; Noxaline “Cdk 1/5 inhibitors”; and/or 4-((5-bromo-2-pyridinyl) amino)-4-oxobutanoic acid “Bibikin”; Li₂CO₃; caffeine; valproic acid, ABC99; LP-922056; or combinations thereof; preferably the Wnt agonist is lithium chloride LiCl, Li₂CO₃; caffeine; or CHIR99021 or combinations thereof.

8. The Wnt agonist for use according to any of claims 3 to 7, wherein the constitutively Wnt antagonist expressing cells decrease the number of functional wild type cells.

9. The Wnt agonist for use according to any of claims 3 to 8, wherein the Wnt agonist renders functional wild type cells insensitive to Wnt antagonist, optionally via downstream Wnt agonist mediated inhibition of GSK3β.

10. The Wnt agonist for use according to any preceding claim, wherein the Wnt agonist is administered simultaneously, separately or sequentially after administration of anti-inflammatory compound, optionally wherein the anti-inflammatory compound is a NSAID; non-NSAID; selective COX-2 inhibitor; or 2-Acetoxybenzoic acid.

11. The Wnt agonist for use according to any preceding claim, wherein the Wnt agonist is administered to the subject at a sub-therapeutic amount in the bloodstream of the subject.

12. The Wnt agonist for use according to claim 11 wherein the Wnt agonist is LiCl or Li₂CO₃ and the subtherapeutic amount in the bloodstream of the subject of LiCl, Li₂CO₃ or a combination thereof is 0.2 mM.

13. A Wnt agonist for use in boosting fitness of wild type cells to prevent gastrointestinal tract cancer in a subject, wherein the subject has a genetic predisposition to gastrointestinal tract cancer.

14. The Wnt agonist for use according to claim 13, wherein the cancer is a stomach, intestinal, colon or rectal cancer.

15. The Wnt agonist for use according to claim 13 or 14, wherein Wnt agonist inhibits the expansion of constitutively Wnt antagonist expressing cells.

16. The Wnt agonist for use according to any one of claims 13-15, wherein the subject has been diagnosed with Familial Adenomatous Polyposis (FAP) or attenuated FAP.

17. The Wnt agonist for use according to any one of claims 13-16 wherein the subject has an APC mutation or a β-catenin mutation, optionally wherein the APC mutation is a germline APC gene mutation.

18. The Wnt agonist for use according to any one of claims 13-17, wherein the Wnt agonist is one or more of: a surrogate wnt; chir; R-Spondin analogue; wnt3a; Wnt 5; Wnt-6a; Norrin; CHIR99021; LiCl; BIO-Acetoxime; CHIR 98014; GSK-3 inhibitor IV; SB216763; SB415286; 5-ethyl-7,8-dimethoxy-1H-pyrrolo [3,4-c]-Isoquinoline-1,3-(2H)-dione “3F8”; 9-bromo-7,12-dihydro-indolo [3,2-d][1 Benzazepine-6 (5H)-one “kenpaullone”; 9-bromo-7,12-dihydro-pyrido [3′, 2′: 2,3 Azepino [4,5-b]indol-6 (5H)-one “1-Azakenpaullone”; N-(3-chloro-4-methylphenyl)-5-(4-nitrophenyl)-1,3,4-oxaxe Diazole-2-amine “TC-G24”; 2-methyl-5-[3-[4-(methylsulfinyl) phenyl]-5-benzofuranyl]-1,3,4-Oxadiazole “TCS 2002”; N-[(4-methoxyphenyl) methyl]-N′-(5-nitro-2-thiazolyl) urea “AR-A014418”; 3-[5-[4-(2-hydroxy-2-methyl-1-oxopropyl)-1-piperazinyl]-2-(trifluoromethyl) phenyl]-4-(1H-indole-3-YI)-1H-pyrrole-2,5-dione “TCS 21311”; 3-[[6-(3-aminophenyl)-7H-pyrrolo [2,3-d]pyrimidin-4-yl]oxy]-phenol “TWS119”; 4-(2-Amino-4-oxo-2-imidazolin-5-ylidene)-2-bromo-4,5,6,7-tetrahydropyrrolo [2,3-c]azepine-8-one “10Z-hymenialdicine”; 1997), 2-[(3-iodophenyl) methylsulfanyl]-5-pyridin-4-yl-1,3,4-oxadiazole “GSK-3 beta inhibitor II”; G 4-benzyl-2-methyl-1,2,4-thiadiazolidine-3,5-dione; FRATtide peptide; 3-amino-1H-pyrazolo [3,4-b]; Noxaline “Cdk 1/5 inhibitors”; and/or 4-((5-bromo-2-pyridinyl) amino)-4-oxobutanoic acid “Bibikin”; Li₂CO₃; caffeine; valproic acid; ABC99; LP-922056; or combinations thereof; preferably the Wnt agonist is lithium chloride LiCl, Li₂CO₃, caffeine; or CHIR99021 or combinations thereof.

19. The Wnt agonist for use according to any of claims 15 to 18, wherein the constitutively Wnt antagonist expressing cells decrease the number of functional wild type cells.

20. The Wnt agonist for use according to any of claims 15 to 18, wherein the Wnt agonist renders functional wild type cells insensitive to Wnt antagonist, optionally via downstream Wnt agonist mediated inhibition of GSK3β.

21. The Wnt agonist for use according to any one of claims 13-20, wherein the Wnt agonist is administered simultaneously, separately or sequentially after administration of anti-inflammatory compound, optionally wherein the anti-inflammatory compound is a NSAID; non-NSAID; selective COX-2 inhibitor; or 2-Acetoxybenzoic acid.

22. The Wnt agonist for use according to any one of claims 13-21, wherein the Wnt agonist is administered to the subject at a sub-therapeutic amount in the bloodstream of the subject.

23. The Wnt agonist for use according to claim 22 wherein the Wnt agonist is LiCl or Li₂CO₃ and the subtherapeutic amount in the bloodstream of the subject of LiCl, Li₂CO₃ or a combination thereof is 0.2 mM.

24. The Wnt agonist for use according to any one of claims 13-23, wherein boosting fitness of wild type cells limits expansion of pre-malignant clones.

25. The Wnt agonist for use according to any one of claims 13-24, wherein the wild type cells interact with mutant cells to confer a competitive advantage to the wild type cells.


SEQUENCE LISTING (50 sequences)

SEQ ID NO: 1 - DNA, 20 bp, Artificial Sequence
  Notum sgRNA1 Exon 1
tcacctcctgctgaacacgt

SEQ ID NO: 2 - DNA, 20 bp, Artificial Sequence
  Notum sgRNA 2 Exon 2
tactatttgaaggagtccaa

SEQ ID NO: 3 - DNA, 20 bp, Artificial Sequence
  Wif1 sgRNA 1 Exon 1
agagccggagcgcgaaagca

SEQ ID NO: 4 - DNA, 20 bp, Artificial Sequence
  Wif1 sgRNA 2 Exon 2
acattctgattgtctcggag

SEQ ID NO: 5 - DNA, 20 bp, Artificial Sequence
  Dkk2 sgRNA 1 Exon 1
atccttgacccgcatcagcg

SEQ ID NO: 6 - DNA, 20 bp, Artificial Sequence
  Dkk2 sgRNA 2 Exon 1
ctcacagctaggcagctcgc

SEQ ID NO: 7 - DNA, 48 bp, Artificial Sequence
  Notum ORF EcoRI Forward primer
gacgacgaattcgccaccatgggaggagaggtgcgcgtgctgctactg

SEQ ID NO: 8 - DNA, 42 bp, Artificial Sequence
  Notum ORF NotI Reverse Primer
gacgacgcggccgcctagttcccattactcagcatccctagc

SEQ ID NO: 9 - DNA, 52 bp, Artificial Sequence
  Wif1 ORF NotI Forward Primer
gacgacgcggccgcgccaccatggctcggagaagagccttccctgctttcgc

SEQ ID NO: 10 - DNA, 45 bp, Artificial Sequence
  Wif1 ORF NotI Reverse Primer
gacgacgcggccgctcaccagatgtaattggattcaggtggatcc

SEQ ID NO: 11 - DNA, 48 bp, Artificial Sequence
  Dkk2 ORF EcoRI Forward Primer
gacgacgaattcgccaccatggccgcgctgatgcgggtcaaggattca

SEQ ID NO: 12 - DNA, 42 bp, Artificial Sequence
  Dkk2 ORF NotI Reverse Primer
gacgacgcggccgctcagatcttctggcatacatggagtctg

SEQ ID NO: 13 - DNA, 25 bp, Artificial Sequence
  qPCR forward primer Mouse Alpi gene
ggctacacacttagggggacctcca

SEQ ID NO: 14 - DNA, 24 bp, Artificial Sequence
  qPCR primer Reverse Mouse Alpi gene
agcttcggtgacattgggccggtt

SEQ ID NO: 15 - DNA, 18 bp, Artificial Sequence
  qPCR forward primer mouse Ascl2 gene
ctactcgtcggaggaaag

SEQ ID NO: 16 - DNA, 19 bp, Artificial Sequence
  qPCR reverse primer Mouse Ascl2 gene
actagacagcatgggtaag

SEQ ID NO: 17 - DNA, 20 bp, Artificial Sequence
  qPCR forward primer Mouse Axin2
ccatgacggacagtagcgta

SEQ ID NO: 18 - DNA, 20 bp, Artificial Sequence
  qPCR reverse primer mouse Axin2
ctgcgatgcatctctctctg

SEQ ID NO: 19 - DNA, 21 bp, Artificial Sequence
  qPCR primer ChgAMouse forward
aagaagaggaggaggaagagg

SEQ ID NO: 20 - DNA, 19 bp, Artificial Sequence
  qPCR primer ChgAMouse Reverse
tccatccactgcctgagag

SEQ ID NO: 21 - DNA, 20 bp, Artificial Sequence
  qPCR primer Dkk2Mouse Forward
tcagtcagccaaccgatctg

SEQ ID NO: 22 - DNA, 22 bp, Artificial Sequence
  qPCR primer Dkk2Mouse reverse
tctctgtggcatcgtttctttt

SEQ ID NO: 23 - DNA, 22 bp, Artificial Sequence
  qPCR primer HprtMouse forward
tgtaatgatcagtcaacggggg

SEQ ID NO: 24 - DNA, 21 bp, Artificial Sequence
  qPCR primer HprtMouse reverse
agaggtccttttcaccagcaa

SEQ ID NO: 25 - DNA, 21 bp, Artificial Sequence
  qPCR primer Krt20Mouse Forward
gctgcaaaatcaggttaagga

SEQ ID NO: 26 - DNA, 22 bp, Artificial Sequence
  qPCR primer Krt20Mouse Reverse
tcccctctcagtctcaaacttc

SEQ ID NO: 27 - DNA, 20 bp, Artificial Sequence
  qPCR primer Lgr5Mouse forward
ttcgtaggcaacccttctct

SEQ ID NO: 28 - DNA, 22 bp, Artificial Sequence
  qPCR primer Lgr5Mouse reverse
tcctgtcaagtgaggaaattca

SEQ ID NO: 29 - DNA, 27 bp, Artificial Sequence
  qPCR primer Lyz1Mouse forward
gccaaggtctacaatcgttgtgagttg

SEQ ID NO: 30 - DNA, 23 bp, Artificial Sequence
  qPCR primer Lyz1Mouse reverse
cagtcagccagcttgacaccacg

SEQ ID NO: 31 - DNA, 20 bp, Artificial Sequence
  qPCR primer Muc2 Mouse forward
ctgaccaagagcgaacacaa

SEQ ID NO: 32 - DNA, 20 bp, Artificial Sequence
  qPCR primer Muc2 Mouse reverse
catgactggaagcaactgga

SEQ ID NO: 33 - DNA, 20 bp, Artificial Sequence
  qPCR primer Notum Mouse forward
ctgcgtggtacactcaagga

SEQ ID NO: 34 - DNA, 20 bp, Artificial Sequence
  qPCR primer Notum Mouse reverse
ccgtccaatagctccgtatg

SEQ ID NO: 35 - DNA, 20 bp, Artificial Sequence
  qPCR primer Rpl37 Mouse forward
ccaaggcctaccaccttcag

SEQ ID NO: 36 - DNA, 20 bp, Artificial Sequence
  qPCR primer Rpl37 Mouse reverse
cagtcccggtagtgtttcgt

SEQ ID NO: 37 - DNA, 20 bp, Artificial Sequence
  qPCR primer Wif1 Mouse Forward
caaagaatgccagccattcc

SEQ ID NO: 38 - DNA, 20 bp, Artificial Sequence
  qPCR primer Wif1 Mouse reverse
cagcaaagggacattgacag

SEQ ID NO: 39 - DNA, 20 bp, Artificial Sequence
  qPCR primer GAPDH Human forward
aatcccatcaccatcttcca

SEQ ID NO: 40 - DNA, 20 bp, Artificial Sequence
  qPCR primer GAPDH Human reverse
tggactccacgacgtactca

SEQ ID NO: 41 - protein, 21 aa, Artificial Sequence
  qPCR primer GUSB Human forward
TGGTTGGAGAGCTCATTTGGA

SEQ ID NO: 42 - DNA, 21 bp, Artificial Sequence
  qPCR primer GUSB Human reverse
gcactctcgtcggtgactgtt

SEQ ID NO: 43 - DNA, 23 bp, Artificial Sequence
  qPCR primer KRT20 Human forward
tcctcaaaaaggagcatcaggag

SEQ ID NO: 44 - DNA, 20 bp, Artificial Sequence
  qPCR primer KRT20 Human reverse
tgatgacgccaaggttcagg

SEQ ID NO: 45 - DNA, 22 bp, Artificial Sequence
  qPCR primer LGR5 Human FW
accagactatgcctttggaaac

SEQ ID NO: 46 - DNA, 21 bp, Artificial Sequence
  qPCR primer LGR5 Human reverse
ttcccagggagtggattctat

SEQ ID NO: 47 - DNA, 20 bp, Artificial Sequence
  qPCR primer MUC2 Human FW
acccgcactatgtcaccttc

SEQ ID NO: 48 - DNA, 20 bp, Artificial Sequence
  qPCR primer MUC2 Human reverse
gcatcatatgcacggtcttg

SEQ ID NO: 49 - DNA, 19 bp, Artificial Sequence
  qPCR primer NOTUM Human FW
actcgcacaggcacaggga

SEQ ID NO: 50 - DNA, 20 bp, Artificial Sequence
  qPCR primer NOTUM Human reverse
gccccgctccaaacatcact